(19) United States
(12) Patent Application Publication
(10) Pub. No.: US 2021/0145730 A1
(43) Pub. Date: May 20, 2021

(54) PROCESS FOR PREPARING AN ORAL DISINTEGRATING DOSAGE FORM

(71) Applicant: Zeenar Enterprises Pty Ltd, Brighton (AU)

(72) Inventors: Tomer MADMON, Melbourne (AU); David KANNAR, Melbourne (AU)

(73) Assignee: Zeenar Enterprises Pty Ltd, Brighton (AU)

(21) Appl. No.: 16/606,167

(22) PCT Filed: Apr. 20, 2018
(86) PCT No.: PCT/AU2018/050364

§ 371 Date: Oct. 17, 2019

(30) Foreign Application Priority Data
Apr. 20, 2017 (AU) 2017901443
Mar. 9, 2018 (AU) 2018900791

(51) Int. Cl. A61K 9/00; A61K 9/20; A61K 31/505; A61K 31/137; A61K 31/40; A61K 31/485

(52) U.S. Cl. CPC A61K 9/006; A61K 9/2013; A61K 9/2095; A61K 31/505; A61K 9/2027; A61K 31/485; A61K 9/2059; A61K 9/2009; A61K 31/137; A61K 31/40; A61K 9/2018

(57) ABSTRACT

An oral disintegrating tablet (ODT) comprising an active ingredient, about 1 to about 20% w/w of at least one amphiphilic compound, about 1 to about 60% w/w of at least one disintegrant and about 0.5 to about 5% w/w of at least one binder, wherein the ODT has hardness of at least about 1 kp and wherein, when the ODT contacts a hydrophilic solvent, the amphiphilic compound self-assembles into liquid crystalline particles.

### FIELD OF THE INVENTION

The present invention relates to oral disintegrating tablets, methods of their manufacture and methods of medical treatment using disintegrating tablets.

### BACKGROUND OF THE INVENTION

An orally disintegrating tablet or orally dissolving tablet (ODT) is a drug dosage form that is a solid oral preparation that disintegrates rapidly in the oral cavity. ODTs differ from traditional tablets in that they are designed to be dissolved in the oral cavity rather than be swallowed whole. For example, an ODT may dissolve on the tongue, sublingually (under the tongue) or buccally (ie on the cheek).

ODTs have been used for patients who experience dysphagia (difficulty in swallowing) and when it is convenient to have a tablet that can be taken without water. Often, these are dosage forms specially designed for children or the elderly.

Drugs delivered via the oral mucosa by ODTs can have a faster onset of effects than drugs delivered by tablets or capsules. Glyceryl trinitrate (GTN) is a known drug that has been formulated as an ODT for rapid drug absorption because rapid absorption is critical to prevent a heart attack. Unfortunately, for other active ingredients rapid onset is unsuitable because the blood concentration of the drug would rise too fast or too far causing side effects and/or resulting in a short therapeutic effect.

ODTs are of interest for drugs that yield low bioavailability through the digestive tract but are inconvenient to administer parenterally, such as steroids and narcotic analgesics. In addition to rapid action, ODT dosage forms may offer significant advantage by avoiding extensive first pass metabolism. This could mean less drug is required to produce an equivalent effect compared to oral administration. Associated side effects may also be reduced. However, ODTs are not currently used where slow release of a drug is required. If speed of ODT absorption could be slowed or managed, then this delivery route could be useful for a wider range of active ingredients.

ODT dosage forms also currently have limited utility for lipophilic drugs, which have difficulty absorbing across the membrane. It would be useful to have an ODT that assists with delivery of lipophilic drugs via oral mucosa.

The excipients used to prepare tablets are generally well known and have been in use for some time. The development of new tableting excipients is relatively rare.

Amphiphilic compounds are often considered unsuitable for use in the preparation of hard tablets. Amphiphilic compounds are often liquids or waxes at room temperature. The properties of amphiphilic compounds are not well suited to solid formulations because they are either liquids or waxes at room temperature and therefore result in unacceptably soft tablets that are not commercially useful.

Amphiphiles are used as excipients in liquid or semi-solid formulations such as emulsions, for example, in shampoos as cleaning agents or to suspend solids in other liquid formulations. Amphiphiles are also used in semi-solid formulations such as creams, for example, glycerol monooleate is generally used as an emulsifier in water-in-oil emulsions. However, amphiphiles have not traditionally been used in solid tablets or ODTs in significant quantities.

A composition comprising an amphiphilic compound capable of self-assembling into liquid crystalline particles that prolongs release of an active ingredient has been described in international patent publication no. WO 2014/179845. However, an ODT was not described in that document. The composition prepared was either a viscous paste or a crumbling wax at room temperature depending on the quantity of the active ingredient niacin.

Reference to any prior art in the specification is not an acknowledgment or suggestion that this prior art forms part of the common general knowledge in any jurisdiction or that this prior art could reasonably be expected to be understood, regarded as relevant, and/or combined with other pieces of prior art by a skilled person in the art.

### SUMMARY OF THE INVENTION

The inventors of the present invention have developed an ODT comprising a new excipient. The excipient is an amphiphile, which forms liquid crystalline particles when the ODT contacts a hydrophilic fluid. In addition, the inventors of the present invention have developed a process for preparing the ODT. The ODT of the invention is an advance over known compositions because, when it disintegrates in a hydrophilic solvent, the amphiphilic compound self assembles into liquid crystalline particles. The new ODT may optionally be used for delivery of active ingredients that were previously unsuitable for ODT delivery. Without being bound by theory, it is thought that the amphiphilic compound encapsulates the active ingredient and assists ODT delivery of active ingredients that are too lipophilic for direct delivery across the mucosal membrane. The ODT of the invention optionally also provides a drug delivery option for active ingredients that are not ideal for oral delivery due to significant first pass metabolism or gastrointestinal side effects. The new ODT optionally also results in prolonged release of some active ingredients via the oral mucosa. Without being bound by theory, this allows delivery of active ingredients via the oral mucosa that cannot be delivered in a traditional ODT because they are absorbed too quickly resulting in side effects or an undesirably short therapeutic effect.

The inventors of the present invention have developed a solid, commercially useful ODT containing an amphiphilic compound. The ODT has suitable tablet hardness, disintegrates rapidly as desired for an ODT, and includes an amphiphilic compound that, when the ODT disintegrates in a hydrophilic solvent, self-assembles into liquid crystalline particles that optionally assist, facilitate or permit delivery of lipophilic active ingredients and/or result in prolonged release of active ingredients.

In one aspect, the present invention provides an ODT comprising an active ingredient, about 1 to about 20% w/w of at least one amphiphilic compound, about 1 to about 60% w/w of at least one disintegrant and about 0.5 to about 5% w/w of at least one binder and wherein, when the ODT contacts a hydrophilic solvent, the amphiphilic compound self-assembles into liquid crystalline particles. Optionally, the ODT has a hardness of about 0.5 to about 6 kp, about 0.5 to about 4 kp, about 1 to about 4 kp or about 1 to about 3 kp, about 1 to about 2 kp or about 1 to about 1.5 kp. Tablet hardness or breaking force is determined according to chapter <1217> of the United States Pharmacopeia on tablet breaking force. Preferably, the amphiphilic compound is glycerol monooleate. Optionally, the amount of binder is 1 to 3% w/w, 1.5 to 2.5% w/w of the ODT. Optionally, the ODT is prepared by wet granulation. Preferably, when the ODT is prepared by wet granulation, the binder is part of a binding solution and the formulation is dried following addition of the binding solution. Optionally the solvent combined with the binder to make binding solution is ethanol. Preferably, the binder is povidone. Preferably, the liquid crystalline particles are cubosomes. Optionally, the ODT is under 500 mg.

The ODT is optionally 5 to 15 mm or 8 to 12 mm in diameter. The ODT is preferred to disintegrate in 15 minutes or less, optionally, in less than 10 minutes and in some embodiments in less than 2 minutes, 1 minute or 30 seconds following contact with a hydrophilic solvent.

Generally, when the ODT of the invention is administered to the oral mucosa, the ODT facilitates systemic administration of the active ingredient across the oral mucosa. In some embodiments, when the ODT disintegrates upon contact with a hydrophilic solvent the amphiphilic compound self-assembles into liquid crystalline particles that encapsulate or entrain the active ingredient.

In a further aspect, the present invention provides a method for confirming that an ODT according to the invention self assembles into liquid crystalline particles following contact with a hydrophilic solvent comprising dissolving an ODT according to the invention in a hydrophilic solvent to produce a suspension and analysing the suspension using the SAXS/WAXS beamline of a synchrotron to determine if liquid crystalline particles are present. Optionally, the exposure time is 5 seconds. Optionally the suspension is prepared at ambient temperature (eg about 22° C.) and the analysis occurs at ambient temperature (eg about 22° C.). The ODT described above can be prepared using the methods described below.

The inventors of the present invention have also developed a method of preparing solid ODTs containing a higher amount of amphiphilic compound than expected to result in a solid tablet given the waxy nature of the amphiphile. Importantly, the solid ODTs of the invention not only have suitable hardness but also incorporate the amphiphilic compound into the ODT in a way that maintains the ability of the amphiphilic compound to self-assemble into liquid crystalline particles. Consequently, when the ODT disintegrates in the presence of a hydrophilic solvent, the amphiphilic compound self-assembles into liquid crystalline particles.

In one aspect, the present invention provides a method of preparing an ODT comprising
heating an amphiphilic compound capable of self-assembly into liquid crystalline particles upon contact with a hydrophilic solvent to its melting point
mixing an active ingredient with the amphiphilic compound until dispersed
cooling the mixture to at least a semi-solid state
combining the mix of active ingredient and amphiphilic compound with at least one pharmaceutically acceptable excipient to prepare a blend
optionally adding further pharmaceutically acceptable excipients
compressing the blend into an ODT

wherein, when the ODT contacts a hydrophilic solvent the ODT disintegrates and the amphiphilic compound self-assembles into liquid crystalline particles.

Optionally, the cooling and combining steps occur simultaneously. Optionally, one or more of the pharmaceutically acceptable excipients is sensitive to acidic environments. Optionally, the amphiphilic compound and the active ingredient are mixed directly ie without any other ingredients present or, alternatively, without significant amounts of any other ingredients. Optionally, the active ingredient is sensitive to acidic environments. Optionally, the mixing of active ingredient and amphiphilic compound comprises dispersing the active ingredient into the amphiphilic compound.

Without being bound by theory, it is thought that mixing the active ingredient directly with the amphiphilic compound that is capable of self-assembly into liquid crystalline particles (without the presence of other ingredients) and cooling the mix before or during addition of further excipients protects the amphiphilic compound from damage due to contact with the excipients when in molten form and preserves the amphiphilic compound's ability to self-assemble into liquid crystalline particles when the formulated ODT contacts a hydrophilic solvent. Similarly, this process minimises degradation of the excipients caused by contact with the amphiphilic compound.

Optionally, the cooling is achieved by combining the mix of active ingredient and amphiphilic compound with the at least one pharmaceutically acceptable excipient in an amount sufficient to reduce the temperature of the amphiphilic compound so that it returns to at least a semi-solid state. The at least one pharmaceutically acceptable excipient is optionally added immediately following mixing of the amphiphilic compound and active ingredient.

Optionally, the heating, mixing and cooling are performed in a way that minimises the amount of time during which the amphiphilic compound is melted. To achieve this, the active ingredient can be mixed with the amphiphilic compound immediately following melting of the amphiphilic compound and the mix of amphiphilic compound and active ingredient can be cooled immediately following sufficient mixing. It is preferred if the mixing of the amphiphilic compound and active ingredient is completed within 30 minutes, 20 minutes, 15 minutes, 10 minutes, 5 minutes, 3 minutes, 2 minutes. Optionally, the mixing is for 0.5 to 2 minutes. It is preferred that the temperature of the amphiphilic compound is controlled so that the amphiphilic compound is not heated more than 10° C., 8° C., 5° C., 3° C. above its melting point. Preferably, the amphiphilic compound is not heated above about 40° C., about 35° C. or about 30° C. Optionally, the amphiphilic compound is heated to about 29-31° C. Preferably, cooling is to about 25° C., about 23° C., about 22° C., about 21° C. or about 20° C.

After combining the homogeneous blend of active ingredient and amphiphilic compound with at least one pharmaceutically acceptable excipient, the ODT may be prepared by direct compression.

Optionally, the blend of active ingredient, amphiphilic compound and at least one pharmaceutically acceptable excipient is granulated (preferably wet granulation) the granules dried and then combined with at least one further pharmaceutically acceptable excipient before compression into an ODT. Where wet granulation is used, ethanol may be used as the solvent and, optionally, one or more excipients may be dispersed or suspended in the ethanol. Once granules are prepared they are optionally blended with further excipients before compression.

Optionally, following preparation of an ODT by the above method, formation of liquid crystalline particles following is confirmed by dissolving the ODT according to the invention in a hydrophilic solvent to produce a suspension and analysing the suspension using the SAXS/WAXS beamline of a synchrotron to determine if liquid crystalline particles are present. Optionally, the exposure time is 5 seconds. Optionally the suspension is prepared at ambient temperature (eg about 22° C.) and the analysis occurs at ambient temperature (eg about 22° C.).

In a further aspect, the present invention provides a method of preparing an ODT comprising
heating an amphiphilic compound capable of self-assembly into liquid crystalline particles upon contact with a hydrophilic solvent to its melting point
mixing an active ingredient with the amphiphilic compound until dispersed immediately following melting of the amphiphilic compound or alternatively during melting the amphiphilic compound
cooling the mixture to at least a semi-solid state immediately following dispersion of the active ingredient within the amphiphilic compound
combining the mix of active ingredient and amphiphilic compound with at least one pharmaceutically acceptable excipient
optionally adding further pharmaceutically acceptable excipients
compressing the blend into one or more ODTs

wherein, when the ODT contacts a hydrophilic solvent the ODT disintegrates and the amphiphilic compound self-assembles into liquid crystalline particles. Optionally, the active ingredient is sensitive to acidic environments. Optionally, the cooling and combining steps occur simultaneously. Optionally, one or more of the pharmaceutically acceptable excipients is sensitive to acidic environments. Optionally, the amphiphilic compound and the active ingredient are mixed directly ie without any other ingredients present or, alternatively, without significant amounts of any other ingredient.

Without being bound by theory, it is thought that minimising the amount of time the active ingredient is in contact with molten amphiphilic compound minimised any detrimental effect of the amphiphilic compound on the active ingredient. It is also thought that minimising the amount of time the amphiphilic compound is molten minimises any detrimental effect on the amphiphilic compound, in particular, any detriment that could prevent the amphiphilic compound from self-assembling into liquid crystalline particles.

Optionally combining the homogeneous blend of active ingredient and amphiphilic compound with at least one pharmaceutically acceptable excipient reduces the temperature of the amphiphilic compound to below its melting point. The at least one pharmaceutically acceptable excipient is optionally added as soon as possible following mixing of the amphiphilic compound and active ingredient.

It is preferred if the mixing of the amphiphilic compound and active ingredient is completed within 30 minutes, 20 minutes, 15 minutes, 10 minutes, 5 minutes or 3 minutes. It is preferred that the temperature of the amphiphilic compound is controlled so that the amphiphilic compound is not heated more than 10° C., 8° C., 5° C., 3° C. above its melting point. Preferably, the amphiphilic compound is not heated above about 40° C., about 35° C. or about 30° C. Preferably, cooling is to about 23° C., about 22° C., about 21° C. or about 20° C.

Optionally, following preparation of an ODT by the above method, formation of liquid crystalline particles following is confirmed by dissolving the ODT according to the invention in a hydrophilic solvent to produce a suspension and analysing the suspension using the SAXS/WAXS beamline of a synchrotron to determine if liquid crystalline particles are present. Optionally, the exposure time is 5 seconds. Optionally the suspension is prepared at ambient temperature (eg about 22° C.) and the analysis occurs at ambient temperature (eg about 22° C.).

During their work, the inventors of the present invention identified a limit to the amount of certain actives and certain excipients that could be directly mixed with the amphiphilic compound glycerol monooleate without altering the ability of the glycerol monooleate to form complex liquid crystalline particles such as cubosomes or hexosomes upon contact with a hydrophilic solvent. Above that limit the mixing of the active ingredient or excipient with the amphiphilic compound had a detrimental effect on the formation complex liquid crystalline particles, that is, lamella particles or no liquid crystalline particles were formed by the amphiphilic compound upon contact with a hydrophilic solvent instead of cubosomes and/or hexosomes.

Following identification of this “direct mixing limit” for certain ingredients, the inventors of the present invention developed a process of preparing an ODT containing amphiphilic compounds that protects the function of the amphiphilic compound, that is, it minimises the risk that additional tabletting ingredients will damage the amphiphilic compound so that complex liquid crystalline particle are not formed when the ODT is contacted by a hydrophilic solvent. This process allows for the inclusion of ingredients in an ODT in an amount above their direct mixing limit without altering the function of the amphiphilic compound.

In some embodiments, the active ingredient and/or at least one excipient are “threshold ingredients”, that is, ingredients that, when directly mixed with the amphiphilic compound at the melting point of the amphiphilic compound for 72 hours, a specific w/w ratio (ie threshold amount) of active ingredient or excipient to amphiphilic compound or more prevents the formation of liquid crystalline particles by the amphiphilic compound upon contact with a hydrophilic solvent. Where the ODT includes a threshold ingredient, the ODT may include more than the threshold amount of that ingredient and the ODT the amphiphilic compound in the ODT retains the ability for form a liquid crystalline phase (or preferably a cubic or hexagonal phase) upon contact with a hydrophilic solvent.

The threshold amount may differ for each active ingredient or excipient and amphiphilic compound combination. In addition, some ingredients may not have a threshold amount. The threshold amount for each active ingredient or excipient with each amphiphilic compound can be determined by combining multiple w/w percentage mixes of active ingredient or excipient with the amphiphilic compound at the melting point of the amphiphilic compound for 72 hours and then testing whether or not the amphiphilic compound forms liquid crystalline phase upon contact with a hydrophilic solvent. Example 1 provides a procedure for assessing formation of a liquid crystalline phase.

Without being bound by theory, it is thought that the ability to include ingredients in the ODT beyond their threshold amounts without preventing formation of liquid crystalline particles (preferably cubic or hexagonal liquid crystalline phase) is because the threshold ingredient is not directly mixed with the molten amphiphilic compound. Instead, the threshold ingredient is added after cooling of the amphiphilic compound to at least a semi-solid state. The threshold ingredient is also added after mixing of the active ingredient and amphiphilic compound. This separation of the molten amphiphilic compound and the threshold ingredient is thought to protect the ability of the amphiphilic compound to form liquid crystalline particles (preferably cubic or hexagonal liquid crystalline phase). Optionally, the blend of active ingredient and amphiphilic compound is cooled so that it is no longer molten or is being cooled to a non-molten state when the threshold ingredient is added to the formulation.

Threshold ingredients include (but are not limited to) tri eithyl citrate (TEC), sodium cyclamate, butylated hydroxy anisole (BHA), saccharin sodium, sodium bicarbonate, menthol, poloxamer 188, and poloxamer 407.

Preferably, the self-assembled structure is a cubic phase or hexagonal phase as discussed below.

In some embodiments, the ODTs of the present invention are prolonged release ODTs. The prolonged release is determined by reference to either an immediate release ODT or an immediate release tablet.

Traditional ODTs are often used for rapid drug delivery, for example, to achieve maximum blood concentration within 30 minutes of administration. An example is the rapid absorption of glyceryl trinitrate or the t(max) for sublingual epinephrine reported in Gu (2002) to be similar to the t(max) for intramuscular injection because the sublingual area provides large mucosal contact surface with high vascularisation that facilitates rapid drug absorption.

An example of an ODT of the invention resulting in prolonged release of an active ingredients is when the active ingredient is a statin, preferably rosuvastatin, and the amphiphilic compound is glycerol monooleate. In that example, the maximum blood concentration of the active ingredient is optionally achieved over 30 minutes following administration of the ODT, over 45 minutes following administration of the ODT, over 1 hour following administration of the ODT, over 2 hours following administration of the ODT, over 3 hours following administration of the ODT, over 4 hours following administration of the ODT or over 5 hours following administration of the ODT. It will be understood that the time taken to maximum blood concentration may vary depending on the active ingredient used.

In some embodiments, the blood concentration following administration of the ODT is within 30% of the maximum blood concentration for 30 minutes, 1 hour, 90 minutes or 2 hours or more.

In all embodiments of the composition of the invention, the amphiphilic compound is capable of self-assembling in to liquid crystalline particles upon contact with a hydrophilic solvent. Therefore, capable of self-assembly into liquid crystalline particles refers to capable of assembling into liquid crystalline particles in hydrophilic solvent of physiological pH, physiological temperature and physiological salinity etc. It is preferred that, as the tablet disintegrates in a hydrophilic solvent the amphiphilic compound self-assembles into liquid crystalline particles. It is preferred that the active ingredient is encapsulated into the liquid crystalline particles. The analytical techniques presently available make it difficult to confirm whether or not the active ingredient is encapsulated within the liquid crystalline particles. It is preferred that the tablet is muccoadhesive, that the liquid crystalline particles are muccoadhesive or both. If the tablet is muccoadhesive, it can adhere to human mucosa, which can be observed when the tablet is administered.

The tablet is for administration to the oral mucosa. One suitable form of administration is sublingual administration (under the tongue). Another suitable form of administration is buccal administration (ie to the buccal vestibule, that is, the area inside the mouth between the lining of the cheek and the teeth/gums). A further form of administration is where the composition is administered under the lip. When administered to the oral mucosa in this way, the active ingredient diffuses into the blood through the mucosa in the mouth.

The amphiphilic compound is optionally present at an amount of about 1 to about 20% or 5 to 20% w/w of the ODT. In some embodiments, the amount of amphiphilic compound is 3 to 10% w/w, 4 to 10% w/w, 7 to 10% w/w, 4 to 8% w/w, 4.5 to 7.5% w/w or 5 to 7% w/w. In preferred embodiments the amount of amphiphilic compound is about 5% w/w or about 7% w/w. Use of about 5% w/w of amphiphilic compound is preferred for formulations with an about 1:1 w/w ratio of amphiphilic compound to active ingredient. Use of about 7% w/w of amphiphilic compound is preferred for formulations with an about 4:1 w/w ratio of amphiphilic compound to active ingredient.

The amphiphilic compound is a compound that possesses both a hydrophilic portion and a hydrophobic portion and is capable of self-assembling into liquid crystalline particles. The amphiphilic compound can also be a mixture of amphiphiles. Amphiphiles capable of self-assembly behaviour have been described in various publications, such as, for example, Drummond (1999). Examples of amphiphiles that are capable of self-assembly include, but are not limited to: surfactants, lipids, and block copolymers. More specifically, the amphiphilic compound is optionally selected from: fatty acids, fatty alcohols, acylglycerols, glycolipids, sphingolipids, phospholipids, cholesterol and mixtures thereof.

Optionally, the amphiphilic compound is non-ionic.

Hydrophilic-lipophilic balance (HLB) is a measure of the hydrophilicity/lipophilicity of an amphiphile. A HLB under 10 indicates lipid solubility and a HLB over 10 indicates water solubility. Optionally, the compound has a HLB of less than about 10, less than 8 or less than 6. Optionally, the HLB is greater than about 1. Optionally, the HLB is 0 to <10, or 1 to <10, 0 to <8, 1 to <8, 0 to <6 or 1 to <6.

The critical packing parameter (CPP) measures the relative volume of the head (hydrophilic portion) and tail (lipophilic portion) of a surfactant. The CPP indicates the type of liquid crystal likely to form when an amphiphilic compound is in solution at a level above its critical micelle concentration. A CPP of 1 means the surfactant is symmetrical. The CPP is the tail volume (V) divided by the sum of the effective head area (a) and the tail length (I) (ie CPP=V/(a.l)). An amphiphile with a CPP <⅓ is likely to form spherical micelles. An amphiphile with a CPP >⅓ but <½ is likely to form cylindrical micelles. An amphiphile with a CPP >½ but <1 is likely to form lamella micelles. An amphiphile with a CPP >1 is likely to form inversed spherical micelles. The amphiphilic compound of the invention optionally has a CPP >⅓, >½, or >1 at body temperature, atmospheric pressure and in water, pbs or saliva.

Optionally, the amphiphilic compound is a non-ionic amphiphile comprising a HLB of 0 to >10 and a CPP of >½. Optionally, the amphiphilic compound is a non-ionic amphiphile comprising a HLB of 1 to >8 and a CPP of >1.

In some embodiments the amphiphilic compound comprises Formula (I):

H-T (I)

wherein

H is selected from the group consisting of an ester, ether, anhydride, amide, amine, carbamide, glycerol, biuret, phenyl, pyridine or phosphate having at least 2 hydrogen bond forming functional groups; and

T is selected form the group consisting of:
(i) a single C₁₂ to C₁₈ alkyl, alkenyl and alkynyl terminally attached to H optionally comprising:
   a. one or more double bonds (preferably cis and at about C7 to C11); or
   b. three or more methyl branches (preferably isoprenoid branching); and
(ii) two C₁₂ to C₁₈ alkyl, alkenyl and alkynyl both terminally attached to H.

The ester and amide groups etc of H can be present in either orientation ie the ester could be —OC(O)-T or —C(O)O-T.

Optionally, the ester, ether, anhydride, amide, amine, carbamide, glycerol, biuret, phenyl, pyridine or phosphate forms part of, or is substituted with, a sugar (eg glucoside), xyloside (monomer or dimer) or C₁ to C₄ alkyl, alkenyl or alkynyl optionally with two to six hydroxyl, amine or methanol groups and attached at either a terminal or non-terminal carbon.

Optionally, H is selected from the group consisting of ester, ether, amine, amide or glycerol.

Optionally, H has 3 to 6 hydrogen bond forming groups.

Optionally, T has a molecular weight of at least >200 amu.

The amphiphilic compound is optionally selected from the group consisting of glycerol monooleate, glyceryl monolinoleate, glyceryl monooleyl ether, oleyl glycerate, monovaccenin, oleyl urea, linoleyl urea, phytanyl urea, hexahydrofarnesyl-urea, monooleain, phytantriol, glucose stearate, fructose stearate and combinations thereof.

In one embodiment, the amphiphilic compound is selected from a fatty acid comprising a 6 to 24 carbon chain, preferably a 12 to 24 carbon chain, more preferably a 16 to 20 carbon chain, most preferably an 18 carbon chain. The amphiphilic compound can also be a mixture of fatty acids. In a preferred embodiment, the amphiphilic compound is selected from one or more mono- and/or di-glycerides of fatty acids comprising a 6 to 24 carbon chain, preferably a 12 to 24 carbon chain, more preferably a 16 to 20 carbon chain, most preferably an 18 carbon chain. The carbon chain may optionally have one or more double bonds such that it is unsaturated. One preferred class of amphiphilic compounds is glycerol monooleates (GMOs). The Handbook of Pharmaceutical Excipients lists GMO as having a HLB of 3.3. In a particularly preferred embodiment the amphiphilic compound is Myverol™ 18-99k (trade mark owned by Kerry Group Services Limited). Myverol™ is generally considered a GMO despite including some non-GMO amphiphiles. Myverol™ 18-99k is produced from the reaction of glycerol with canola (low erucic acid rapeseed) oil and contains a mixture of monoacylglycerols, diacylglycerols and glycerol. The compositional analysis of Myverol™ 18-99k is detailed in Clogston (Clogston 2000) wherein Myverol™ 18-99k was found to contain 82% monoacylglycerols (consisting of 86.6% monoolein (1-Oleoyl-rac-glycerol), 7.0% monostearin (1-Stearoyl-rac-glycerol), 3.5% monopalmitin (1-monohexadecanoyl-rac-glycerol), 0.9% monoarachidin (1-Arachidonoyl-glycerol) and 2.0% unidentified monoacylglycerols), 13.4% diacylglycerols (consisting of 7.4% 1,2-diacylglycerol and 6.0% 1,3-diacylglycerol) and 4.3% glycerol.

Another grade of GMO suitable for use in the present invention is comprised of about 90-100% monoglycerides (preferably about 95%), about 0-10% diglycerides (preferably about 4%) and about 0-2% triglycerides (preferably about 0.5%). It is preferred if this GMO has not less than 60% methyl oleate (preferably about 65%) and more preferred that the GMO also has not more than 35% methyl linoleate (preferably about 18-20%). The remaining fatty acid composition of the GMO is optionally not more than 12% methyl palmitate (preferably about 4%), not more than 6% methyl stearate (preferably about 2%), not more than 2% methyl linolenate, not more than 2% methyl arachidate, not more than 2% methyl eicosenate and not more than 6% free glycerine (preferably less than 1%).

Thus, in one embodiment the amphiphilic compound is a mixture of amphiphiles. Preferably, the amphiphilic compound contains a mixture of monoacylglycerols, diacylglycerols and glycerol. In particular, the mixture of amphiphiles is produced by reacting glycerol with canola oil. One suitable available amphiphilic compound contains 82% monoacylglycerols, 13.4% diacylglycerols and 4.3% glycerol. More particularly, the amphiphilic compound can contain:

82% monoacylglycerols consisting of 86.6% monoolein, 7.0% monostearin, 3.5% monopalmitin, 0.9% monoarachidin and 2.0% unidentified monoacylglycerols;

13.4% diacylglycerols consisting of 7.4% 1,2-diacylglycerol and 6.0% 1,3-diacylglycerol; and 4.3% glycerol.

In a further embodiment, the amphiphilic compound includes (i) a mixture of a mono- and/or di-glyceride of one or more fatty acids and (ii) one or more free fatty acids. Thus, the amphiphilic compound may include Myverol™ 18-99k and a fatty acid, such as oleic acid. In a further embodiment, the amphiphilic compound includes monoacylglycerol and oil.

The self-assembled particles are optionally selected from the following group: cubosomes, hexosomes, sponge particles and mixtures thereof, preferably cubosomes.

The self-assembled particles may form a bulk phase selected from the group consisting of micellar (normal and reversed), lamellar, hexagonal (normal and reversed), cubic (normal discrete, reversed discrete, reversed bicontinuous—including primitive, gyroid and diamond—and reversed discontinuous), and other ‘intermediate phases’ such as the ribbon, mesh, or non-cubic ‘sponge’ bicontinuous phases. See Israelachvili, J (1994), Chang (1998) and Kaasgard (2006) for more detail. In a preferred embodiment, the bulk phase is selected from cubic phase, hexagonal phase and mixtures thereof, preferably reversed bicontinuous cubic phase, preferably the diamond phase. Optionally, the bulk phase is lamellar phase. Optionally, the bulk phase is lamellar, reversed cubic or reversed hexagonal.

Without being bound by theory or mode of action, it is believed that the more complex the self-assembled particles and/or bulk phase, the slower the release of the active ingredient. Thus, the hexagonal and cubic, particularly diamond cubic, bulk phases are believed to result in the slowest release.

In one embodiment, the active ingredient is incorporated or dissolved within the self-assembled structure. Preferably, the active ingredient is non-covalently incorporated. The active ingredient is optionally in the form of a prodrug. In this embodiment, the active ingredient needs to be cleaved, for example by an enzyme or hydrolysis, either before or after absorption to form the active ingredient.

In preferred embodiments, the pharmaceutically acceptable disintegrant is present at an amount of about 1 to about 60% w/w of the ODT. In some embodiments the amount of pharmaceutically acceptable disintegrant is about 10 to about 50%, about 20 to about 60%, about 20 to about 50%, about 20 to about 40%, about 15 to about 40% w/w, about 20 to about 30% w/w, about 10 to about 20% w/w of the ODT. Alternatively, the ODT is prepared with low disintegrant content of about 1 to about 10% w/w of the ODT.

In some embodiments, the pharmaceutically acceptable disintegrant is selected from the group consisting of sodium starch glycolate, copovidone, crosslinked polyvinylpyrrolidone (crospovidone) or a derivative of crospovidone such as, crosslinked sodium carboxymethyl cellulose (croscarmellose sodium) sodium/calcium carboxymethylcellulose, sodium bicarbonate, microcrystalline cellulose, low-substituted hydroxypropylcellulose or sodium starch glycolate. It is possible to add crospovidone to the ODT independently or in the form of a blend such as Pharmaburst™, which contains 7-15% crospovidone.

Preferred formulations of the invention comprise two or more disintegrants. It is preferred if one of the two or more disintegrants is crospovidone. In some embodiments, the two or more disintegrants include both crospovidone and sorbitol copovidone. In some embodiments there are two disintegrants. These disintegrants are optionally crospovidone and copovidone or sodium starch glycolate and crospovidone.

Preferred formulations of the invention comprise three or more disintegrants. The three or more disintegrants are preferred to be crospovidone, copovidone and sodium starch glycolate.

Where the formulation includes crospovidone, the crospovidone is preferred to be about 5 to about 45%, about 10 to about 45% w/w of the formulation, about 15 to about 35% w/w of the formulation or about 20 to about 25% w/w of the formulation. Where the formulation includes crospovidone, and sodium starch glycolate, the preferred amounts of crospovidone are as above and the preferred amount of sodium starch glycolate is about 3 to about 8% w/w or about 5% w/w of the formulation.

In some embodiments, w/w ratio of amphiphilic compound to active ingredient is about 1:1 or more, about 4:1 or more, or about 7:1 or more, about 10:1 or more, about 50:1 or more or about 100:1 or more. Alternatively, the w/w ratio of amphiphilic compound to active ingredient is about 1:1, about 4:1 or about 7:1. It is also possible for the ratio of amphiphilic compound to active ingredient to be lower than 1:1, for example, 1:1.5, 1:2 or 1:3, particularly where the active ingredient has good water solubility. Higher ratios of amphiphilic compound to active ingredient are more likely where the dose of active ingredient is low.

It is preferred for the composition to adhere to the oral mucosa. Alternatively or in addition, the self-assembled structures formed upon contact with a hydrophilic solvent are preferred to adhere to oral mucosa. Optionally, the solvent is saliva or water.

In some embodiments of the present invention, the composition physically disintegrates into particles upon contact with a hydrophilic solvent (such as saliva).

The ODT is suitable for administration to oral mucosa. An ODT suitable for administration to the oral mucosa comprises an active ingredient, an amphiphile, a disintegrant and optionally a muccoadhesive. In some embodiments, disintegration occurs in 2 to 20 minutes, 2 to 15 minutes or 2 to 10 minutes. Alternatively, disintegration takes less than 120 seconds, 1 to 110 seconds, 10 to 90 seconds, 60 seconds or less, 30 seconds or less or 10 seconds or less. In other embodiments, disintegration takes 3 to 5 minutes, 5 to 10 minutes or 5 to 15 minutes. In some embodiments ODTs are formulated for speed of disintegration and others are formulated to enhance permeation of the mucosa. For example, an enhancer may be included to improve permeation but this could slow disintegration and stabilizing polymers such as polyethylene glycol could be avoided to speed disintegration. In muccoadhesive embodiments the muccoadhesive is optionally the same ingredient as the amphiphile. Some amphiphiles have muccoadhesive properties, for example, glycerol monooleate.

All embodiments of the composition of the invention may further comprise additional pharmaceutically acceptable excipients such as one or more filler, binder, glidant, lubricant, osmotic agent, sweetener and/or flavour.

The ODT may further include a second active ingredient. It is preferred for the active ingredient and optional second active ingredient in the ODT to be micronized. The particle size of the active ingredient and optional second active ingredient in the ODT is preferred to be about 10 μm.

In some embodiments, the active ingredient is hydrophilic. In alternate embodiments, the active ingredient is lipophilic.

Optionally, the active ingredient has a log P of −0.5 to 6.4. Optionally, the active ingredient has a molecular weight of 100 to 1,200 g/mol. Optionally, the active ingredient has both a log P of −0.5 to 6.4 and a molecular weight of 100 to 1,200 g/mol.

In some embodiments, the active ingredient is about 0.05% to about 10% w/w or about 0.1% to about 6% w/w of the ODT. Optionally, the ratio of GMO to active ingredient is about 1:1 to 4:1 by weight.

Suitable active ingredients include statins, niacin, amoxicillin, clavulanic acid, trimethoprim, sulfamethoxazole, 5HT2c anti-serotonins, phenteramine, beta blockers, thiazide diuretics, steroids, ACE inhibitors, aspirin, paracetamol and ibuprophen or their derivatives. Other suitable active ingredients include oxycodone, adrenaline ie epinephrine, melatonin, atenolol, irinotecan, paclitaxel, atropine, haloperidol, levofloxacin, indomethacin, diazepam, trans retinol, prednisolone, progesterone, hydrocortisone, dexamethasone, delta-9-tetrahydrocannabinol, paracetamol/acetaminophen and capecitabine. Suitable statins include, but are not limited to: atorvastatin, fluvastatin, lovastatin, mevastatin, pitavastatin, pravastatin, rosuvastatin, simvastatin and mixtures thereof. Statins used in the invention are optionally hydrophobic for a statin (ie lipophilic) but are preferably hydrophilic for a statin. Hydrophilic statins, including fluvastatin, pravastatin and rosuvastatin are less toxic than lipophilic statins, including atorvastatin, lovastatin and simvastatin. In a preferred embodiment, the pharmaceutical composition may further include any one or more of fluvastatin, pravastatin and rosuvastatin.

In one embodiment, the active ingredient is rosuvastatin and the w/w active ingredient in the ODT is optionally 10 to 50%, 20-50%, 30-50%, 40-50% or about 50%.

As with all ODTs, it is apparent that the ingredients in the ODT of the invention should be selected so that there is no negative interaction between the active ingredient and the excipients. The excipients should be selected so that they are compatible with the active ingredient and the amphiphilic compound and the ODT is stable for at least 3 or 6 months. In one embodiment, the active ingredient is rosuvastatin, the amphiphilic compound is glycerol monooleate and the ODT is stable for at least 2 years, at least 1 year, at least 9 months, at least 6 months or at least 3 months. More specifically, the ODT of this embodiment is optionally stable at 25° C./60% relative humidity for at least 2 years, at least 1 year, at least 9 months, at least 6 months or at least 3 months and/or stable at 5° C. for at least 2 years, at least 1 year, at least 9 months, at least 6 months or at least 3 months, in particular, preferred ODTs retain about 90% or about 95% or more active ingredient following storage at either 25° C./60% relative humidity or 5° C. for at least 2 years, at least 1 year, at least 9 months or at least 6 months. Optionally, there is also no change in lactone or 5-oxo-rosuvastatin calcium levels following storage at 5° C. for at least 6 months and/or less than 0.5% w/w lactone and less than 0.4% w/w 5-oxo-rosuvastatin calcium following storage at 25° C./60% relative humidity for at least 6 months.

All embodiments of the ODT of the invention may further comprise additional pharmaceutically acceptable excipients such as one or more filler, binder, glidant, lubricant, osmotic agent, sweetener and/or flavour.

An ODT may be combined with a hydrophilic solvent prior to administration or administered along with a hydrophilic solvent.

Glycerol monooleate has been shown to be a suitable amphiphile. Each of the above forms of the invention includes a form where the amphiphilic compound is glycerol monololeate.

The present invention has a number of specific forms. Additional embodiments are of these forms are as discussed elsewhere in the specification. The following aspects of the invention further describe options for administration of the ODT. For example, in a further aspect the present invention provides an ODT comprising an active ingredient, about 1 to about 20% w/w of at least one amphiphilic compound, about 1 to about 60% w/w of at least one disintegrant and about 0.5 to about 5% w/w of at least one binder, wherein, when the ODT contacts a hydrophilic solvent, the amphiphilic compound self-assembles into liquid crystalline particles and the ODT has a hardness of about 0.5 to about 6 kp and is less than about 500 mg,

In a further aspect the present invention provides an ODT comprising an active ingredient, about 1 to about 20% w/w of at least one amphiphilic compound, about 1 to about 60% w/w of at least one disintegrant and about 0.5 to about 5% w/w of at least one binder, wherein, when the ODT contacts a hydrophilic solvent, the amphiphilic compound self-assembles into liquid crystalline particles and the liquid crystalline particles are cubosomes.

In a further aspect the present invention provides an ODT comprising an active ingredient, about 1 to about 20% w/w of at least one amphiphilic compound, about 1 to about 60% w/w of at least one disintegrant and about 0.5 to about 5% w/w of at least one binder, wherein, when the ODT contacts a hydrophilic solvent, the amphiphilic compound self-assembles into liquid crystalline particles and the liquid crystalline particles are lamella.

In a further aspect, the present invention provides an ODT comprising an active ingredient, about 1 to about 20% w/w of at least one amphiphilic compound, about 1 to about 60% w/w of at least one disintegrant and about 0.5 to about 5% w/w of at least one binder, wherein, when the ODT contacts a hydrophilic solvent, the amphiphilic compound self-assembles into liquid crystalline particles and the ODT disintegrates in a hydrophilic solvent in less than 2 minutes.

In a further aspect, the present invention provides a method of preparing an ODT comprising
heating an amphiphilic compound capable of self-assembly into liquid crystalline particles upon contact with a hydrophilic solvent to its melting point
mixing an active ingredient with the amphiphilic compound until dispersed
cooling the mixture to at least a semi-solid state
combining the mix of active ingredient and amphiphilic compound with at least one pharmaceutically acceptable excipient to prepare a blend
optionally adding further pharmaceutically acceptable excipients
compressing the blend into an ODT

wherein, when the ODT contacts a hydrophilic solvent the ODT disintegrates and the amphiphilic compound self-assembles into liquid crystalline particles and the amphiphilic compound and the active ingredient are mixed directly by dispersing the active ingredient in the amphiphilic compound.

In a further aspect, the present invention provides a method of preparing an ODT comprising
heating an amphiphilic compound capable of self-assembly into liquid crystalline particles upon contact with a hydrophilic solvent to its melting point
mixing an active ingredient with the amphiphilic compound until dispersed
cooling the mixture to at least a semi-solid state
combining the mix of active ingredient and amphiphilic compound with at least one pharmaceutically acceptable excipient to prepare a blend
optionally adding further pharmaceutically acceptable excipients
compressing the blend into an ODT

wherein, when the ODT contacts a hydrophilic solvent the ODT disintegrates and the amphiphilic compound self-assembles into liquid crystalline particles, the amphiphilic compound is not heated above 40° C. and the cooling is achieved by combining the mix of active ingredient and amphiphilic compound with the at least one pharmaceutically acceptable excipient in an amount sufficient to reduce the temperature of the amphiphilic compound so that it returns to at least a semi-solid state.

In a further aspect, the present invention provides a method of preparing an ODT comprising
heating an amphiphilic compound capable of self-assembly into liquid crystalline particles upon contact with a hydrophilic solvent to its melting point
mixing an active ingredient with the amphiphilic compound until dispersed
cooling the mixture to at least a semi-solid state
combining the mix of active ingredient and amphiphilic compound with at least one pharmaceutically acceptable excipient to prepare a blend
optionally adding further pharmaceutically acceptable excipients
compressing the blend into an ODT wherein, when the ODT contacts a hydrophilic solvent the ODT disintegrates and the amphiphilic compound self-assembles into liquid crystalline particles and the ODT comprises a threshold ingredient in an amount above its direct mixing limit.

In a further aspect, the present invention provides a method of preparing an ODT comprising
heating an amphiphilic compound capable of self-assembly into liquid crystalline particles upon contact with a hydrophilic solvent to its melting point
mixing an active ingredient with the amphiphilic compound until dispersed immediately following melting of the amphiphilic compound or alternatively during melting the amphiphilic compound
cooling the mixture to at least a semi-solid state immediately following dispersion of the active ingredient within the amphiphilic compound
combining the mix of active ingredient and amphiphilic compound with at least one pharmaceutically acceptable excipient
optionally adding further pharmaceutically acceptable excipients
compressing the blend into one or more ODTs

wherein, when the ODT contacts a hydrophilic solvent the ODT disintegrates and the amphiphilic compound self-assembles into liquid crystalline particles, the mixing of the amphiphilic compound and active ingredient is completed within 30 minutes and the amphiphilic compound is not heated above 40° C.

In a further aspect, the present invention provides a method of preparing an ODT comprising
heating an amphiphilic compound capable of self-assembly into liquid crystalline particles upon contact with a hydrophilic solvent to its melting point
mixing an active ingredient with the amphiphilic compound until dispersed immediately following melting of the amphiphilic compound or alternatively during melting the amphiphilic compound
cooling the mixture to at least a semi-solid state immediately following dispersion of the active ingredient within the amphiphilic compound
combining the mix of active ingredient and amphiphilic compound with at least one pharmaceutically acceptable excipient
optionally adding further pharmaceutically acceptable excipients
compressing the blend into one or more ODTs

wherein, when the ODT contacts a hydrophilic solvent the ODT disintegrates and the amphiphilic compound self-assembles into liquid crystalline particles, the mixing of the amphiphilic compound and active ingredient is completed within 30 minutes and the ODT comprises a threshold ingredient in an amount above its direct mixing limit.

wherein the active ingredient has a log P of −0.5 to 6.5.

wherein the amphiphilic compound has a critical packing parameter (CPP) of >½ and/or a hydrophilic lipophilic balance (HLB) of 0 to >10. Optionally, the CPP is >1 and the HLB is 1 to <8.

These methods of preparing an ODT may be used to prepare the ODTs of the invention, as described elsewhere in the specification.

In a further aspect, the present invention provides a method for confirming that an ODT according to the invention self assembles into liquid crystalline particles following contact with a hydrophilic solvent comprising dissolving an ODT according to the invention in a hydrophilic solvent to produce a suspension and analysing the suspension using the SAXS/WAXS beamline of a synchrotron to determine if liquid crystalline particles are present. Optionally, the exposure time is 5 seconds. Optionally the suspension is prepared at ambient temperature (eg about 22° C.) and the analysis occurs at ambient temperature (eg about 22° C.). In some embodiments, when the ODT disintegrates upon contact with a hydrophilic solvent the amphiphilic compound self-assembles into liquid crystalline particles that encapsulate or entrain the active ingredient.

This methods may be used to test the ODTs of the invention, as described elsewhere in the specification.

As used herein, except where the context requires otherwise, the term “comprise” and variations of the term, such as “comprising”, “comprises” and “comprised”, are not intended to exclude further additives, components, integers or steps.

Further aspects of the present invention and further embodiments of the aspects described in the preceding paragraphs will become apparent from the following description, given by way of example and with reference to the accompanying drawings.

### BRIEF DESCRIPTION OF THE DRAWINGS

FIG. 1—Outline of the lyotropic liquid crystalline phases that can be formed when water is added to an anhydrous lipid. Normal (o/w) phases are designed I and inversed (w/o) phases II with decreasing packing parameter as water concentration increase. Adapted with permission from Israelachivili et al. (1994) and Nguyen (2009). Israelachvili, J., The science and applications of emulsions—an overview. Colloids Surf. Physico chem. Eng. Aspects 1994, 91, 1-8. Nguyen, T.-H. Investigation of novel liquid crystalline materials for the sustained oral delivery of poorly water soluble drugs. PhD, Monash University, Melbourne, 2009.

FIG. 2—Diagrammatic representation of the structure of the three main bicontinuous and hexagonal crystal structures where (a) Gyroid (G) Ia3d, (b) Diamond (D) Pn3m and (c) Primitive (P) Im3m. The hexagonal liquid crystals are represented by (d) inverse and (e) normal hexagonal structure. Diagram adapted from Caffrey and Cheng (1995) and Nguyen (2009).

FIG. 3—The appearance of oxycodone in the receptor chamber over time after application of SBT227 (diamond) and SBT232 (square) to porcine buccal mucosa in the donor chamber of a Ussing chamber. Data are presented as mean±SEM (n=5). Both formulations demonstrate slow release characteristics. SBT227 release the API relatively faster comparing to SBT232 due to lower quantity of GMO.

FIG. 4—The appearance of atorvastatin in the receptor chamber over time after application of SBT226 (square) and SBT233 (diamond) to porcine buccal mucosa in the donor chamber of a Ussing chamber. Data are presented as mean±SEM (n=5). Both formulations demonstrate slow release characteristics. SBT226 release the API relatively faster comparing to SBT233 due to lower quantity of GMO.

FIG. 5—The results for each individual tablet that were combined to prepare the mean results depicted in FIG. 4 are shown in FIG. 5A—SBT226 tablet testing and FIG. 5B—SBT233 testing. Release of atorvastatin into the receptor chamber at 0, 0.25, 0.5, 1, 1.5, 2, 3 and 4 hours is shown. The individual testing results demonstrate slow release continuing at 4 hours for 4 out of the 5 SBT226 tablets and 3 out of the 5 SBT233 tablets.

### DETAILED DESCRIPTION OF THE EMBODIMENTS

It will be understood that the invention disclosed and defined in this specification extends to all alternative combinations of two or more of the individual features mentioned or evident from the text or drawings. All of these different combinations constitute various alternative aspects of the invention.

Reference will now be made in detail to certain embodiments of the invention. While the invention will be described in conjunction with the embodiments, it will be understood that the intention is not to limit the invention to those embodiments. On the contrary, the invention is intended to cover all alternatives, modifications, and equivalents, which may be included within the scope of the present invention as defined by the claims.

Further aspects of the present invention and further embodiments of the aspects described in the preceding paragraphs will become apparent from the following description, given by way of example.

All of the patents and publications referred to herein are incorporated by reference in their entirety.

For purposes of interpreting this specification, terms used in the singular will also include the plural and vice versa.

One skilled in the art will recognize many methods and materials similar or equivalent to those described herein, which could be used in the practice of the present invention. The present invention is in no way limited to the methods and materials described.

The inventors of the present invention have developed a process for preparing an ODT that prolongs release of an active ingredient. The prolonged release is achieved by including in the ODT an amphiphilic compound capable of self-assembly into liquid crystalline particles upon contact with a hydrophilic solvent.

The inventors have also developed an ODT of suitable hardness despite including significant quantities of amphiphiles normally reserved for semi-sold formulation. Not only does the ODT have suitable hardness but it also incorporate the amphiphilic compound into the ODT in a way that maintains the ability of the amphiphilic compound to self-assemble into liquid crystalline particles

The ingredient GMO has traditionally been used in the preparation of hard tablets. The inventors of the present invention have also prepared oral disintegrating tablets of pharmaceutically acceptable hardness comprising significant quantities of the ingredient GMO.

Certain ingredients at certain amounts interfere with the formation of liquid crystalline particles by the amphiphilic compound, either by preventing formation of said particles or by altering the type of particles formed. The method of the present invention allows use of ingredients that interfere with formation of the liquid crystalline particles at amounts that would otherwise be an issue. For example, the inventors of the present invention identified that mixes of the amphiphilic compound glycerol monooleate (GMO) with the active ingredient rosuvastatin were a problem at more than 15% rosuvastatin to GMO (w/w). Using the process of the invention, an ODT containing 50% rosuvastatin to GMO (w/w) was prepared and the GMO retained the ability to form liquid crystalline cubic phase.

The term ‘self-assembled particles’ as used throughout the specification is understood to mean an aggregate of amphiphiles that possess some degree of internal organisational order, for example, a colloidal particle or colloidosome or a solid lipid particle. The particles can be either nanoparticles or microparticles depending on their average size, typically less than about 1 μm, preferably in a range of about 10 nm to about 500 nm, more commonly about 200 nm. Solid lipid nanoparticles are a dispersed crystalline lamellar lipidic material. The self-assembled particles are formed by contacting the amphiphile with solvent. In some embodiments, the self-assembled particles themselves aggregate into a bulk lyotropic phase.

The term ‘bulk phase’ as used throughout the specification is understood to mean a lyotropic phase that includes but is not limited to: micellar cubic (11); normal hexagonal (H1); bicontinuous cubic (V1); lamellar (L); reversed bicontinuous cubic (V2); reversed hexagonal (H2); reversed micellar cubic (12) and sponge (L3) phases.

The term ‘cubic phase’ as used throughout the specification is understood to refer to two main classes of phases: micellar cubic and bicontinuous cubic. ‘Micellar cubic phase’ refers to a phase consisting of spherical micelles arranged in a cubic array. A ‘normal micellar cubic phase’ or ‘II phase’ consists of spherical normal micelles arranged in a cubic array, whilst an ‘inverse micellar cubic phase’ or ‘III phase’ consists of spherical inverse micelles arranged in a cubic array. ‘Bicontinuous cubic phase’ refers to a family of closely related phases that consist of a single curved lipid bilayer that forms a complex network that separates the polar solvent space into two continuous, but non-intersecting volumes. Bicontinuous cubic phases possess long range order based upon a cubic unit cell. Bicontinuous cubic phases have zero mean curvature; that is, at all points on surface of the amphiphile bilayer, the surface is as convex as it is concave. Bicontinuous cubic phases include the normal (‘vI phase’) or reverse (‘vII phase’) type. Several types of long range orientational orders have been observed for bicontinuous cubic phases; the orientational order in these phases correspond to space groups Ia3d, Pn3m, and Im3m. When a colloidosome possesses the internal structure of a bulk cubic phase the colloidosome is referred to as a ‘cubosome’.

The term ‘hexagonal phase’ as used throughout the specification is to be understood to mean an amphiphile phase consisting of long, rod-like micelles packed into a hexagonal array. A ‘normal hexagonal phase’ is a hexagonal phase consisting of long, rod-like normal micelles, whilst an ‘inverse hexagonal phase’ is a hexagonal phase consisting of long, rod-like inverse micelles. The normal hexagonal phase may be referred to as the ‘HI phase’ and the inverse hexagonal phase may be referred to as the ‘HII phase’. When a colloidosome possesses the internal structure of a bulk hexagonal phase the colloidosome may be referred to as a ‘hexosome’.

The term ‘lamellar phase’ as used throughout the specification is to be understood to mean a stacked bilayer arrangement, where opposing monolayers of the hydrophilic portion of amphiphile molecules are separated by a polar solvent domain, while the hydrophobic portion of the amphiphile molecule of the back-to-back layers are in intimate contact to form a hydrophobic layer. The planar lamellar phase is referred to as the ‘Lα phase’. There are three lamellar phases, (1) the fluid lamellar phase (Lα) where the chains are melted, (2) the gel lamellar phase (Lß) where the chains are mostly melted but some degree of short range order and (3) the lamellar crystalline phase (Lc), where the chains are crystalline with very short range order.

The term ‘sponge phase’ or ‘L3 phase’ as used throughout the specification refers to a phase that resembles a bicontinuous cubic phase, in that it possesses an amphiphile bilayer that separates the polar solvent space into two unconnected volumes, but it does not possess long range order. Accordingly, these phases are analogous to a ‘melted cubic phase’.

The term ‘prodrug’ as used throughout the specification refers to a biologically active agent including structural modifications thereto, such that in vivo the prodrug is converted, for example, by hydrolytic, oxidative, reductive or enzymatic cleavage to the biologically active agent by one or more reactions or steps. It includes an agent that requires one or more chemical conversion steps or steps of metabolism to produce the active molecule.

The term ‘pharmaceutical composition’ as used throughout the specification means a composition comprising a therapeutically effective amount of at least one active ingredient according to the current invention. The pharmaceutical composition may further include one or more of a pharmaceutically acceptable carrier, excipient, diluent, additive or vehicle selected based upon the intended form of administration, and consistent with conventional pharmaceutical practices. Suitable pharmaceutical carriers, excipients, diluents, additives and vehicles are known to those skilled in the art and are described in publications, such as, for example Remington (Remington: The Science and Practice of Pharmacy, 21st Ed, University of the Sciences in Philadelphia (eds), Lippincott Williams & Wilkins, Philadelphia, Pa., 2005.).

As used herein, ‘therapeutically effective amount’ relates to the amount or dose of a statin or other active thereof that will lead to one or more desired effects, in particular the reduction of cholesterol synthesis. A therapeutically effective amount of a statin will vary according to factors such as the disease state, age, sex, and weight of a subject, and the ability of the substance to elicit a desired response in the subject.

Liquid Crystalline Particles

There are multiple forms of liquid crystalline particles each with different structure. The self-assembled structure may be micellar (normal or reversed), lamellar, hexagonal (normal or reversed), cubic (normal discrete, reversed discrete, reversed bicontinuous—including primitive, gyroid and diamond—or reversed discontinuous), or other ‘intermediate phases’ such as the ribbon, mesh, or non-cubic ‘sponge’ bicontinuous phases. When these particles possess the internal structure of a reversed bicontinuous cubic phase, the particles are colloquially referred to as cubosomes. Similarly, when the particles possess the internal structure of a reversed hexagonal phase, they are referred to as hexosomes. When the particles possess the internal structure of a lamellar phase, they are referred to as liposomes.

The type of phase structures formed is dependent on the amphiphile structure, amphiphile concentration, temperature, pressure and solvent content (Kaasgaard, T.; Drummond, C. J., Ordered 2-D and 3-D nanostructured amphiphile self-assembly materials stable in excess solvent. Phys. Chem. Chem. Phys. 2006, 8, 4957-4975). The relationship between phase structures formation and the geometry of amphiphilic molecules can be defined by the dimensionless critical packing parameter (p)=v/al where v and I are the volume and the length of the fully extended hydrocarbon chain and a is the surface area of the hydrophilic headgroup. An increase in a may occur due to an increase in hydration and electrostatic repulsion between adjacent hydrophilic headgroups. Whilst an increase in v can be due to increases in hydrocarbon chain fluidity at elevated temperature66 or increases in the number, branching and/or size of the hydrocarbon chain. At p=1, which indicates equal hydrophilic and hydrophobic volume, lamellar (La) bilayer structure are formed. Normal (Type 1) self-assembled structure with the interface curves spontaneously towards water (positive curvature) are formed when p<1 whilst inverse structures (Type 2) with interface curves spontaneously away from water (negative curvature) are formed when p >1. In addition to micellar (p<⅓), inverse micellar (p >3) and lamellar structures, other phases such as the two dimensional normal and inverse hexagonal (H1 and H2), the three dimensional normal and inverse bicontinuous cubic (V1 and V2) and the discontinuous cubic (I1 and I2) phases are also observed (structure to be discussed in detail later). It should be noted that a range of nomenclatures are used in literature to denote the individual phase in the literature, so for this report, the abbreviations just mentioned will be utilised.

Lamellar, hexagonal and cubic phases are considered as liquid crystal phases as they exhibit both the long range order of crystalline materials and the disorder of liquid systems.

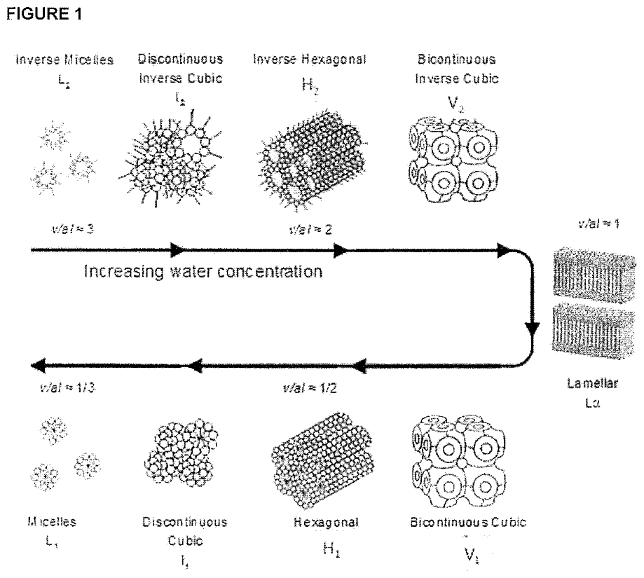

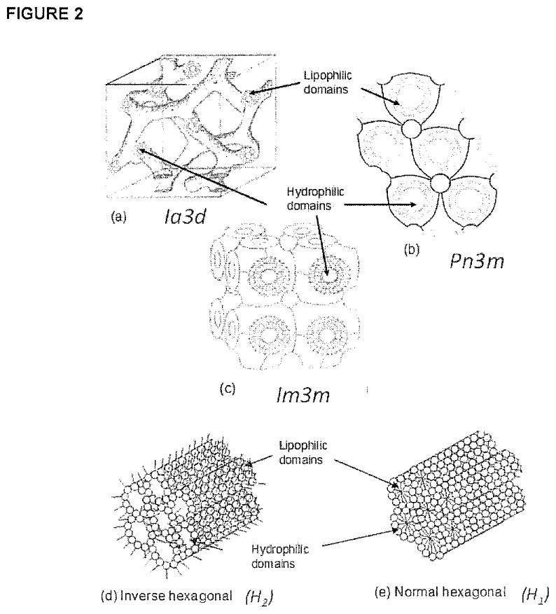

For conventional surfactants, a progressive increase in the water content induces changes in the phase structure from inverse phases to normal phases as illustrated in FIG. 1.1. Not all structures illustrated in FIG. 1.1 may be observed, but the order and progression of the general phase change is preserved.

However, some amphiphilic molecules do not follow the illustrated lyotropic phase behaviour in FIG. 1.1 all the way to micelles in addition water. These molecules only swell in contact with water to a finite degree and forms liquid crystalline phases depending on their chemical structure, and further addition of water does not induce the transition to micelles. Hence these inverse LC phases co-exist with excess water at very high dilution. The most well-known example of this behaviour is phospholipids which remain as La phase in excess water and are the critical component of cellular membranes.

Lamellar Liquid Crystals

Lamellar liquid crystal (LC) phases consist of stacked bilayers, where lipid molecules are arranged so that the hydrophobic chains meet to form a hydrophobic domain whilst the hydrophilic head groups facing opposite ends form the hydrophilic domains with other lamellae bilayers. Water occupies the hydrophilic domain and interacts with the hydrophilic head groups lining each lamellae. The lamellar liquid crystals are formed when the geometric space occupied by the hydrophilic headgroup and the hydrophobic tail are equivalent (packing parameter 1). Lamellar structures are the arguably the most ubiquitous liquid crystal structure of all lyotropic liquid crystals as they are featured in most biological membranes.

Cubic Liquid Crystals

Micellar Cubic Structures (I)

Discontinuous cubic phases (as opposed to the bicontinuous cubic phases discussed later) are micelles embedded in a three-dimensional, matrix organised in a cubic symmetry. The discontinuous cubic phases, whether normal (I1) or inverted (I2) are intermediate LC phases and reside between hexagonal LCs and micelles in the order of progression described in FIG. 1.

Bicontinuous Cubic Liquid Crystals (V)

Bicontinuous cubic LC phases, whether normal V1 or inverse V2 are viscous, optically isotropic liquid crystals located on either side of the lamellar structure and differ from the discontinuous cubic phases, as they consists of separate but continuous lipid bilayer and water regions.

The inverse bicontinuous cubic phase (V2) consists of two separate, continuous but nonintersecting hydrophilic regions divided by a single lipid bilayer in a complex 3-D cubic symmetry. It is generally believed that V2 phases have interface structures based on the infinite periodic minimal surfaces (IPMS), where the lipid surface consists of two principle curvatures which are equal but opposite in sign at every point (as convex as they are concave), resulting in zero average mean curvature (positive+negative curvature), and negative Gaussian curvature (positive×negative curvature). Using X-ray scattering measurements the existence of cubic phases in amphiphile+water systems was recognized in the 60s. Although mathematicians have found a wide variety of periodic minimal surfaces, only three types of IPMS have been observed in amphiphile-water systems: Gyriod (G), double diamond (D) and primitive (P) with corresponding space groups of Ia3d (G), Pn3m (D) and Im3m (P), respectively (see FIG. 1). The different phases can be identified by their unique X-ray scattering patterns.

Hexagonal Liquid Crystals (H)

Hexagonal LCs are designated as either H1 (normal) or inverse H2 phases. The H phases are viscous, optically birefringent liquid crystals consisting of infinitely long hexagonally close packed cylindrical micelles (see FIG. 1). The H2 phase has more negative curvature than the inverse cubic phase due to larger hydrophobic portion of the molecules.

Bulk Phase

The liquid crystalline particles of the present invention may self-assembled into bulk phase including an active ingredient. Typically, a bulk material having a certain phase will form from an amphiphile, that is, a molecule that possesses both a hydrophilic portion and a hydrophobic portion. The self-assembly behaviour of amphiphiles in solvent arises because of the preferential interaction between the solvent and either the hydrophilic or hydrophobic portion of the amphiphilic molecule. When an amphiphile is exposed to a polar solvent, the hydrophilic portion of the amphiphile tends to preferentially interact with the polar solvent, resulting in the formation of hydrophilic domains. The hydrophobic portion of the amphiphile molecules tend to be excluded from this domain, resulting in the de facto formation of a hydrophobic domain.

It is in a self-assembled form that amphiphiles are capable of acting as an inert carrier or matrix into which biologically active molecules, such as an active ingredient, may be incorporated. The nanoscale porosity of the self-assembled materials provides a high internal and external surface area. An active ingredient that is distributed within a region of this material is believed to be distributed in an ordered arrangement, and at a high loading concentration due to the large internal and external liquid crystal surface area. Self-assembled bulk phase may exhibit a variety of orientational orders. If long-range orientational order is observed within the self-assembled bulk phase at equilibrium, the self-assembled bulk phase is termed a ‘mesophase’, a ‘lyotropic liquid crystalline phase’, a ‘lyotropic phase’ or, as used herein, simply a ‘phase’.

There are 2 principal types of liquid crystalline phases: thermotropic liquid crystals and lyotropic liquid crystals. Thermotropic liquid crystals can be formed by heating a crystalline solid or by cooling an isotropic melt of an appropriate solute. Lyotropic liquid crystals can be formed by addition of a solvent to an appropriate solid or liquid amphiphile. The manipulation of parameters such as amphiphile concentration and chemical structure, solvent composition, temperature and pressure may result in the amphiphile-solvent mixture adopting lyotropic phases with distinctive characteristics.

Examples of particular phases that can be formed by self-assembled particles are set out above. It is possible to disperse the bulk phases described above to form colloidal particles (so-called ‘colloidosomes’) that retain the internal structure of the non-dispersed bulk phase. When these particles possess the internal structure of a reversed bicontinuous cubic phase, the particles are colloquially referred to as cubosomes. Similarly, when the particles possess the internal structure of a reversed hexagonal phase, they are referred to as hexosomes. When the particles possess the internal structure of a lamellar phase, they are referred to as liposomes.

Whilst the bulk materials can be of use in some circumstances, the use of bulk materials having cubic phases in drug administration is limited by their high viscosity making them difficult to administer. In these cases, colloidal dispersions of particles of these phases may be used in drug delivery. More preferred phases for use as drug delivery vehicles are bicontinuous cubic phase or reversed hexagonal phase. The inverse cubic phase affords distinct aqueous regions that form two continuous water networks (or channels) throughout the cubic phase that more readily allow diffusion of an active ingredient. The inverse cubic liquid crystal phase is thermodynamically stable and co-exists in equilibrium with excess water over a broad temperature range. Alternatively, if the bicontinuous cubic phase is viscous and difficult to administer it may be possible to administer a lamellar phase material that converts into the cubic phase upon dissolution with aqueous, water rich, body fluids (thus facilitating the conversion of one phase to another). For example, a suitable material is a phospholipid such as 1,2-dioleoyl-sn-glycero-3-phosphocholine. The cubic phase in situ provides a viscous depot from which an active ingredient can slowly be released. An inverse cubic liquid crystal phase provides an appropriate scaffold in which to distribute or load the niacin compound owing to the high surface area of the internal liquid crystal structure (up to 400 m²/g).

One of the key difficulties with using liquid crystalline particles in the formulation of dosage forms for active ingredient delivery has been the viscosity of many liquid crystalline phases, which are difficult to handle, difficult to manufacture and difficult to administer.

Suitable pharmaceutical carriers, excipients, diluents, additives and vehicles are known to those skilled in the art and are described in publications, such as, for example Remington (Remington: The Science and Practice of Pharmacy, 21st Ed, University of the Sciences in Philadelphia (eds), Lippincott Williams & Wilkins, Philadelphia, Pa., 2005.).

The formulation may include one or more binders such as hydroxypropylmethylcellulose (HPMC), ethyl cellulose, acacia, polyvinyl alcohol (PVA), and polyvinylpyrrolidone (Povidone).

The formulation may include one or more glidants such as talc, magnesium trisilicate and colloidal silicon dioxide.

The formulation may include one or more fillers such as lactose, mannitol, sorbitol, starch, maltodextrin, acacia and silicon dioxide.

The formulation may include one or more lubricants such as glyceryl behenate, stearic acid, talc, zinc stearate, calcium stearate, magnesium stearate, aluminium stearate and sodium stearyl fumarate.

The formulation may include one or more film formers such as hydroxypropylmethylcellulose (HPMC), povidone (PVP), poly ethylene glycol (PEG).

The formulation may include one or more pH modifier agents (buffering agents) such as sodium hydroxide, sodium/calcium carbonate, citric acid, tartaric acid, fumaric acid etc.

If the formulation is prepared by thermoplastic granulation the formulation may include thermoplastic granulation agents such as glycerol monostearate, and glyceryl behenate.

The presence of liquid crystalline phase can be determined using the SAX/WAX beamline of a synchrotron, cross polarised light microscopy (CPLM) or Cry-Em. In certain circumstances, such as a low proportion of amphiphilic compound in the ODT, liquid crystalline phase may not be identified using the SAX/WAX beamline of the synchrotron and an alternative, such as, CPLM may be preferred. CPLM can identify LC structures but does not provide information on the internal phase.

The CPP of an amphiphilic compound can be determined by quantum mechanics molecular simulations to determine geometrical and quantitative structure-activity relationship (QSAR) values. See, Fong 2016. The HLB of an amphiphilic compound is calculated based on the number and identity of hydrophilic/lipophilic groups.

The CPP and HLB of some amphiphilic compounds are in Table A.

TABLE A
CPP and HLB for various amphiphilic compounds

| Amphiphile | CPP | V | A₀ | L_{C} | HLB |
|---|---|---|---|---|---|
| Phytantriol | 0.650 | 303.5 | 27.9 | 16.8 | 6.36 |
| Monolinolein | 1.016 | 341.0 | 22.6 | 14.8 | 1.02 |
| Glucose | 0.456 | 315.3 | 31.2 | 22.2 | 9.28 |
| stearate | | | | | |
| Fructose | 0.421 | 315.3 | 33.8 | 22.2 | 9.28 |
| stearate | | | | | |

The active ingredients melatonin and atenolol have been shown to load and release from a monoolein-water liquid crystalline system previously and are expected to be compatible with the ODT of this invention. Atropine, haloperidol, levofloxacin, indomethacin, diazepam, trans retinol, prednisolone, progesterone, hydrocortisone and dexamethasone have been shown to load an release from monoolein and/or phytantriol liquid crystals previously and are expected to be compatible with the ODT of this invention. Irinotecan and paclitaxel has also been released from inverse hexagonal phase previously and are expected to be compatible with the ODT of this invention.

The log P and molecular weight for some active ingredients are in Table B below.

TABLE B
LogP and molecular weight for various active ingredients

| API | MW (g/mole) | logP |
|---|---|---|
| Atorvastatin | 1155.4 | 6.36 |
| Oxycodone | 351.9 | 0.3 |
| Adrenaline | 183.2 | −0.5 to −1.2 |
| Rosuvastatin | 1001.1 | 1.80 at pH 5 |
| | | 0.30 at pH 7 |
| Niacin | 123.1 | 0.36 |

### REFERENCES

The text of each of the following references is incorporated by reference into this specification.
Caffrey, M.; Cheng, A., Kinetics of lipid phase changes. Curr. Opin. Struct. Biol. 1995, 5, 548-555.
Chang, C.-M.; Bodmeier, R., Low viscosity monoglyceride-based drug delivery systems transforming into a highly viscous cubic phase. Int. J. Pharm. 1998, 173, 51-60.
Clogston, J.; Rathman, J.; Tomasko, D.; Walker, H.; Caffrey, M., Phase behavior of a monoacylglycerol (Myverol 18-99K)/water system. Chem. Phys. Lipids 2000, 107, 191-220.
Drummond, C. J.; Fong, C., Surfactant self-assembly objects as novel drug delivery vehicles. Current Opinion in Colloid & Interface Science 1999, 4, 449-456.
Fong, W et al, Dynamic formation of nanostructured particles from vesicles via invertase hydrolysis for on-demand delivery, The Royal Society of Chemistry: Electronic Supplementary Material (ESI) for RSC Advances, 2016, S1-S22.
Gu, X et al, Is epinephrine administration by sublingual tablet feasible for the first-aid treatment of anaphylaxis? A proof-of-concept study. Biopharm Drug Dispos. 2002, 23(5):213-6.
Hyde, S. T., Bicontinuous structures in lyotropic liquid crystals and crystalline hyperbolic surfaces. Current Opinion in Solid State and Materials Science 1996, 1, 653-662.
Israelachvili, J., The science and applications of emulsions—an overview. Colloids Surf. Physico chem. Eng. Aspects, 1994, 91, 1-8.
Kaasgaard, T.; Drummond, C. J., Ordered 2-D and 3-D nanostructured amphiphile self-assembly materials stable in excess solvent. Phys. Chem. Chem. Phys. 2006, 8, 4957-4975).
World Intellectual Property Office publication, WO 2014/179845.

### Example 1—Testing Mixtures of GMO and Excipients/Active Ingredients for Effect on Formation of Liquid Crystal Particles

GMO and excipient or active ingredient at various weight proportions were added into small HPLC vials. To ensure adequate mixing, the samples were initially heated above the melting temperature of GMO (>40° C.) and mixed vigorously with a metal spatula. The samples were then mixed via a roller mixer at ˜10 RPM at 40° C. for at least 3 days.

One week prior to SAXS experiment, 100 mg samples were loaded into a transparent polystyrene 96 well plate (Nunc™) and immersed in 200 μL of PBS buffer (pH 6.8). The samples were stored away from light at ambient temperature, to allow the samples to reach equilibrium.

Small Angle x-Ray Scattering (SAXS) Setup

The SAXS/WAXS beamline at Australian Synchrotron, Melbourne, Australia was used to determine the liquid crystalline nanostructure in the samples.

A custom-designed plate holder was used to mount the samples plate directly onto the SAXS/WAXS beamline. Scans were automated using a pre-loaded set of position variables based on the well positions within the plate, the exposure time was 5 sec.

Data were obtained at ambient temperature (˜22° C.). The experiments used a beam of wavelength λ=1.033 Å (12.0 keV) and a typical flux of 1.2×1013 photons/s. The 2-D diffraction images were recorded on a Pilatus 1M detector and radially integrated using the in-house software “ScatterBrain”.

The liquid crystal phase structures were determined by indexing the Bragg peaks according to their corresponding reflection laws (see Hyde, S. T., Bicontinuous structures in lyotropic liquid crystals and crystalline hyperbolic surfaces. Current Opinion in Solid State and Materials Science 1996, 1, 653-662).

The results in tablet 1 to 3 below show GMO forming a bulk phase of complex crystalline particles (ie cubic or hexagonal or a mix of the two) following mixing with a number of ingredients and then emersion of the mix in a hydrophilic solvent (PBS buffer). However, when the ratio of ingredient to GMO increases, the GMO ceases to form a complex bulk phase of crystalline particles but instead forms simple lamella or reverse lamella phase. The percentage w/w of the ingredient to GMO at which complex bulk phase no longer forms is the “threshold” for the ingredient or the maximum amount of the ingredient that can be present before formation of a complex bulk phase is disrupted.

TABLE 1
Summary of liquid crystalline phase formation for mixes of GMO
and another ingredient

| | % w/w | Liquid crystalline phase |
|---|---|---|
| Ingredient | ingredient | formed |
| Tri ethyl citrate (TEC) | up to 20% | cubic |
| | 35% and above | reverse emulsion |
| Sodium cyclamate | up to 10% | cubic |
| | 20% | lamellar |
| | 50% | lamellar + crystals |
| Butylated hydroxy | 1% | cubic |
| anisole (BHA) | | |
| | 5% | cubic + hexagonal |
| | 10-20% | hexagonal |
| | 30% and above | reverse emulsion |
| Sacharin sodium | up to 10% | cubic |
| | 20% and above | mostly lamellar small amount |
| | | cubic |
| Sodium bicarbonate | 1% | Mostly Cubic. Hexagonal in small |
| | | concentration |
| | 5-10% | Hexagonal |
| | 20% | Reversed micellular cubic and |
| | | lamellar |
| | 35% and above | Reverse emulsion |
| Menthol | 1% | Cubic |
| | 5-10% | Hexagonal |
| | 20% and above | Lamellar |
| Poloxamer 188 | Up to 10% | Cubic |
| | 20-35% | Mix of cubic and Lamellar |
| | Above 35% | Lamellar |
| Poloxamer 407 | Up to 10% | Cubic |
| | 20-35% | Mix |
| | Above 35% | Lamellar |
| Rosuvastatin | up to 15% | Cubic (2 types) |
| | 20% and above | Lamellar |

TABLE 2
More detail on the liquid crystalline phase formation for mixes of
GMO and another ingredient

| Concentration | Phase | Phase structure | Phase structure for | Phase |
|---|---|---|---|---|
| in | structure for | for sodium | butylated hydroxy | structure for |
| GMO (%) | TEC | cyclamate | anisole (BHA) | saccharin sodium |
| 1 | V2(Pn3m) (ie | V2(Pn3m) | V2(Pn3m) | V2(Pn3m) |
| | cubic) | | | |
| 5 | V2(Pn3m) | V2(Pn3m) | V2(Pn3m) + H2 | V2(Pn3m) |
| 10 | V2(Pn3m) | V2(Pn3m) + other | H2 | V2(Pn3m) + others |
| 15 | | | H2 | — |
| 20 | V2(Pn3m) | Lα (lamella) | H2 | Lamellar |
| 35 | L2 (reverse | Lα + other | L2 | Unknown |
| | lamella) | | | |
| 50 | L2 | Lα + other | L2 | Unknown |

TABLE 3
More detail on the liquid crystalline phase formation for mixes of
GMO and another ingredient continued

| Concentration | Phase structure | Phase structure for | Phase structure for | Phase structure for |
|---|---|---|---|---|
| in GMO (%) | for menthol | poloxamer 188 | poloxamer 407 | rosuvastatin |
| 1 | V2(Pn3m) | V2(Pn3m) + V2(Im3m) | V2(Pn3m) + V2(Im3m) | V2(Pn3m) |
| 5 | H2 | V2(Im3m) | V2(Im3m) | V2(Pn3m) |
| 10 | H2 | V2(Im3m) | V2(Im3m) | V2(Pn3m) |
| 15 | — | — | — | V2(Pn3m) + V2(Im3m) |
| 20 | L2 | Mixed | V2(Im3m) + Lα | Lα |
| 35 | L2 | Lα | Lα | Lα + Lα |
| 50 | L2 | Lα | Lα | Lα |

### Example 2—Preparing an ODT

Process for wet granulation manufacturing of ODT of Table 4:
GMO was melted (about 30° C.)
As soon as possible following melting of the GMO, rosuvastatin calcium was dispersed in melted GMO for the minimum time needed (about 5 minutes)
Povidone and any menthol & sacharin sodium was dissolved in ethanol
Any mannitol, pharmaburst, sodium chloride, sodium cyclamate and crospovidone were wet granulated with the Rosuvastatin:GMO suspension and the Povidone solution. The addition of these further ingredients lowered the temperature of the GMO so that it returned to a solid or semisolid form (for example to 25° C.)
The granules were dried and milled
The milled granules were blended with the remaining excipients using high shear
The final blend was compressed into ODT tablets

### Example 3—ODT with Increased Rosuvastatin to GMO Proportion

Example 1 shows that when GMO and rosuvastatin are directly mixed there is a 15% limit of inclusion for rosuvastatin before GMO is prevented from forming a cubic liquid crystalline structure upon contact with a hydrophilic solvent.

Using the process of Example 2 and the ingredients in Table 4 below, an ODT with 50% rosuvastatin to GMO has been developed. The GMO has retained the ability to form cubic liquid crystalline structures.

TABLE 4
5 mg statin formulation with 50% rosuvastatin to GMO (SBT122)

| Ingredient | % w/w | Function |
|---|---|---|
| Mannitol | 75.3 | Filler/Carrier |
| Rosuvastatin Calcium | 7.3 | Drug substance/API |
| Povidone (Poly vinyl pyrrolidone) | 1.7 | Binder |
| Glyceryl Monooleate (GMO) | 7.3 | Bioadhesive/Mucoadhesive |
| | | agent, Gelling agent, nonionic |
| | | surfactant, sustained release |
| | | agent |
| Sodium Starch Glycolate | 5.0 | Disintegration agent |
| Colloidal Silicon Dioxide | 2.0 | Glidant |
| Magnesium Stearate | 1.5 | Lubricant |
| Ethanol | N/A * | Solvent |

* Evaporated during the drying process.

Disintegration testing was conducted in a basket-rack assembly and in accordance with Appendix XII A. Disintegration of the European Pharmacopoiea edition 9.0 (Ph. Eur. Method 2.9.1). The solvent was water at 37° C.

The ODT of Table 4 disintegrated within 8-10 minutes and achieved 100% dissolution in water within 15 minutes (Dissolution apparatus II, paddles, 50 rpm, 900 ml, Citrate buffer pH 6.6).

Using the process of Example 2 and the ingredients in Table 5 below, another ODT with 50% rosuvastatin to GMO has been developed. The GMO has retained the ability to form cubic liquid crystalline structures.

TABLE 5
5 mg statin ODT with 50% rosuvastatin to rosuvastatin/GMO total
(SBT176)

| Ingredient | % w/w | Function |
|---|---|---|
| Pharmaburst-(co-processed | 68.67 | Filler, Taste masking, |
| mixture of Mannitol, Sorbitol | | Disintegration agent. |
| Crospovid one & Silicon dioxide) | | |
| Crospovidone XL | 15.00 | Disintegration agent |
| Sodium Chloride | 0.25 | Osmotic agent |
| Sodium Cyclamate | 0.60 | Sweetener |
| Saccharin Sodium | 0.40 | Sweetener |
| Menthol (Optional) | 0.20 | Flavouring agent |
| Rosuvastatin Calcium | 5.44 | Drug substance/API |
| Povidone (Poly vinyl | 1.50 | Binder |
| pyrrolidone) | | |
| Glyceryl Monooleate (GMO) | 5.44 | Bio adhesive/Mucoadhesive |
| | | agent, Gelling agent, nonionic |
| | | surfactant, sustained release |
| | | agent |
| Sodium Starch Glycolate | 5.00 | Disintegration agent |
| Colloidal Silicon Dioxide | 1.50 | Glidant |
| Magnesium Stearate | 1.00 | Lubricant |
| Ethanol | N/A * | Solvent |

* Evaporated during the drying process.

ODT preparation is according to example 2 except mixing of the rosuvastatin calcium and GMO is for 0.5-1 minute.

The tablets weighed 100 mg, had a hardness of 1.3-2.1 kp, friability of 0.1%, 2.05 mm thickness and disintegrated in 30 seconds following emersion in a hydrophilic solvent.

8 mm round tablets of this formulation were stability tested at 5° C. for 9 months. Assay of the 5 mg rosuvastatin showed 97.1% at t=0, 98.1% at t=3 months, 95.8% at t=6 months and 96% at t=9 months. The formulation was also stability tested at 25° C./60% RH for 9 months. Assay of the 5 mg rosuvastatin showed 97.1% at t=0, 94.2% at t=3 months, 96.1% at t=6 months and 94.4% at t=9 months. In addition, the assay of the tablets showed 0.03% at t=0, 0.16% at t=3 months, 0.20% at t=6 months and 0.34% at t=9 months of rosuvastatin in the lactone form and 0.15% at t=0, 0.25% at t=3 months, 0.29% at t=6 months and 0.34% at t=9 months of 5-oxo-rosuvastatin calcium (TP-13 impurity 1) at following storage at 25° C./60% RH. The assay of the tablets also showed 0.03% at t=0, 0.03% at t=3 months, 0.03% at t=6 months and 0.06% at t=9 months of rosuvastatin in the lactone form and 0.15% at t=0, 0.18% at t=3 months, 0.17% at t=6 months and 0.21% at t=9 months of the 5-oxo-rosuvastatin calcium at both 3 and 6 months at 5° C.

The structure of 5-oxo-rosuvastatin calcium is below.

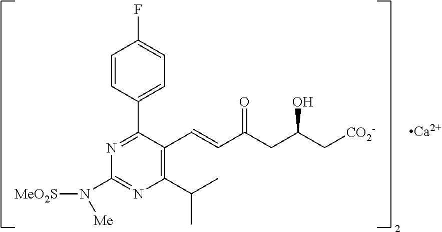

Using the process of Example 2 and the ingredients in Table 6 below, an ODT with 20% rosuvastatin to GMO has been developed. The GMO has retained the ability to form cubic liquid crystalline structures.

TABLE 6
5 mg statin ODT with 20% rosuvastatin to GMO (SBT177)

| Ingredient | % w/w | Function |
|---|---|---|
| Pharmaburst-(co-processed | 64.47 | Filler, Taste masking, |
| mixture of Mannitol, Sorbitol | | Disintegration agent. |
| Crospovid one & Silicon dioxide) | | |
| Crospovidone XL | 15.00 | Disintegration agent |
| Sodium Chloride | 0.60 | Osmotic agent |
| Sodium Cyclamate | 0.25 | Sweetener |
| Saccharin Sodium | 0.40 | Sweetener |
| Menthol | 0.20 | Flavouring agent |
| Rosuvastatin Calcium | 1.81 | Drug substance/API |
| Povidone (Poly vinyl | 2.50 | Binder |
| pyrrolidone) | | |
| Glyceryl Monooleate (GMO) | 7.26 | Bio adhesive/Mucoadhesive |
| | | agent, Gelling agent, nonionic |
| | | surfactant, sustained release |
| | | agent |
| Sodium Starch Glycolate | 5.00 | Disintegration agent |
| Colloidal Silicon Dioxide | 1.50 | Glidant |
| Magnesium Stearate | 1.00 | Lubricant |
| Ethanol | N/A * | Solvent |

* Evaporated during the drying process.

ODT preparation is according to example 2 except mixing of the rosuvastatin calcium and GMO is for 1-2 minutes.

The tablets weighed 300 mg, had a hardness of 2.4-3.5 kp, friability of 0.1%, 2.65 mm thickness and disintegrated in 60-90 seconds following emersion in a hydrophilic solvent.

The ODTs of Tables 5 and 6 achieve 100% dissolution within 5 minutes (Dissolution apparatus II, paddles, 50 rpm, 900 ml, Citrate buffer pH 6.6).

The ODTs of Tables 4, 5 and 6 were each dissolved in hydrophilic solvent and analysed by the SAXS/WAXS beamline at Australian Synchrotron. The GMO of each tablet form cubic liquid crystalline phase.

The tablets were administered sublingually to three different human subjects (one Caucasian male, one Caucasian female and one Asian male) and the speed of tablet disintegration monitored. The formulation in Table 6 disintegrated within 20 to 40 seconds of administration for all three subjects. The formulation in Table 2 disintegrated within 40 to 90 seconds of administration for all three subjects.

The manufacturing of tablets involved mixing of Rosuvastatin with the GMO at its melting point for a short period, until a homogenous dispersion was obtained (approximately 5 minutes) and then mixed with other excipients using a high shear mixer. When combined with the other excipients the temperature of GMO returned to below the GMO melting point and the GMO returned to its semi-solid form.

The formulation of Table 4 disintegrated within 20-40 seconds of contact with oral mucosa (Basket-rack assembly, Ph. Eur. Method 2.9.1, water at 37° C.). The formulation of Table 4 disintegrated within 40-90 seconds of contact with oral mucosa.

12 mm round tablets of this formulation were stability tested at 5° C. for 6 months. Assay of the 5 mg rosuvastatin showed 100.1% at t=0, 100.4% at t=3 months and 97.9% at t=6 months. The formulation was also stability tested at 25° C./60% RH for 6 months. Assay of the 5 mg rosuvastatin showed 100.1% at t=0, 98.6% at t=3 months and 97.6% at t=6 months. In addition, the assay of the tablets showed 0.05% at t=0, 0.31% at t=3 months and 0.49% at t=6 months of rosuvastatin in the lactone form and 0.3% at t=0, 0.31% at t=3 months and 0.36% at t=6 months of 5-oxo-rosuvastatin calcium (TP-13 impurity 1) at following storage at 25° C./60% RH. The assay of the tablets also showed 0.05% at t=0, 0.08% at t=3 months and 0.05% at t=6 months of rosuvastatin in the lactone form and 0.3% at t=0, 0.23% at t=3 months and 0.24% at t=6 months of the 5-oxo-rosuvastatin calcium at both 3 and 6 months at 5° C.

Using the process of Example 2 and the ingredients in Table 7 below, another ODT with 50% rosuvastatin to GMO has been developed. The GMO has retained the ability to form cubic liquid crystalline structures.

TABLE 7
5 mg statin ODT with 50% rosuvastatin to rosuvastatin/GMO total
and 42.5% menthol to menthol/GMO total (SBT131)

| Ingredient | % w/w | Function |
|---|---|---|
| Mannitol BP | 74.9 | Carrier |
| Sodium Starch Glycolate | 5.0 | Disintegration agent |
| Menthol | 4.0 | Flavoring/cooling agent |
| Rosuvastatin Calcium | 5.4 | Drug substance/API |
| (micronized) | | |
| Povidone (Poly vinyl | 1.7 | Binder |
| pyrrolidone) | | |
| Glyceryl Monooleate (GMO) | 5.4 | Bioadhesive/Mucoadhesive |
| | | agent, Gelling agent, nonionic |
| | | surfactant, sustained release |
| | | agent |
| Ethanol | N/A | Solvent |
| Colloidal Silicon Dioxide | 2.0 | Glidant |
| Magnesium Stearate | 1.5 | Lubricant |

* Evaporated during the drying process.

Example 1 shows that when GMO and menthol are directly mixed 1% menthol results in cubic liquid crystalline structure, 5% menthol results in hexagonal liquid crystalline structure and 20 to 50% results in reverse lamella crystalline structure upon contact with a hydrophilic solvent.

Using the process of Example 2 and the ingredients in Table 8 below, another ODT with 50% rosuvastatin to GMO has been developed. The GMO has retained the ability to form cubic liquid crystalline structures.

TABLE 8
5 mg statin ODT with 50% rosuvastatin to rosuvastatin/GMO total
and 48% sodium bicarbonate to sodium bicarbonate/GMO total (SBT127)

| Ingredient | % w/w | Function |
|---|---|---|
| Mannitol BP | 73.9 | Carrier |
| Sodium Starch Glycolate | 5.0 | Disintegration agent |
| Sodium Bicarbonate | 5.0 | Alkalizing agent |
| Rosuvastatin Calcium | 5.4 | Drug substance/API |
| (micronized) | | |
| Povidone (Poly vinyl pyrrolidone) | 1.7 | Binder |
| Glyceryl Monooleate (GMO) | 5.4 | Bioadhesive/Mucoadhesive |
| | | agent, Gelling agent, nonionic |
| | | surfactant, sustained release |
| | | agent |
| Ethanol | N/A | Solvent |
| Colloidal Silicon Dioxide | 2.0 | Glidant |
| Magnesium Stearate | 1.5 | Lubricant |

Example 1 shows that when GMO and menthol are directly mixed 1% sodium carbonate results in cubic and hexagonal liquid crystalline structure, 5% sodium carbonate results in hexagonal liquid crystalline structure and 35% and above results in reverse lamella crystalline structure upon contact with a hydrophilic solvent.

### Example 7—Confirming Formation of Liquid Crystalline Phase

Preparation of Samples Tablets for Simulated Dissolution Study

For equilibrium samples, the tablets were loaded into a transparent polystyrene 96 well plate (Nunc™) and immersed in PBS buffer (pH 6.8). The samples were stored away from light at ambient temperature overnight prior to SAXS experiment.

For the kinetic study of SBT122 and SBT123, 2 tablets were carefully placed in a transparent polystyrene 24 well plate (2 ml per well) to ensure the X-ray beam can transmit through the tablets. The X-ray diffraction patterns were first taken without any solution added. The tablets were then immersed in 2 mL of PBS buffer or saliva donated by the author and other volunteers, the X-ray diffraction patterns were taken overtime, up to 4 hours.

For the kinetic study of SBT177, 2 tablets were disintegrated in PBS using 2 ml vials, the 0.4 ml carefully placed in a transparent polystyrene 96 well plate (0.4 ml per well). The X-ray diffraction patterns were taken overtime, up to 4 hours.

Small Angle X-Ray Scattering (SAXS) Setup

The SAXS/WAXS beamline at Australian Synchrotron, Melbourne, Australia was used to determine the liquid crystalline nanostructure in the samples.

A custom-designed plate holder was used to mount the samples plate directly onto the SAXS/WAXS beamline. Scans were automated using a pre-loaded set of position variables based on the well positions within the plate, the exposure time was 5 seconds. For the kinetic study of SBT177 a single location was tested rather than a full scan of the well.

Data were obtained at ambient temperature (˜22° C.). The experiments used a beam of wavelength λ=1.033 Å (12.0 keV) and a typical flux of 1.2×1013 photons/s. The 2-D diffraction images were recorded on a Pilatus 1M detector and radially integrated using the in-house software “ScatterBrain”.

The liquid crystal phase structures were determined by indexing the Bragg peaks according to their corresponding reflection laws (see Hyde, S. T., Bicontinuous structures in lyotropic liquid crystals and crystalline hyperbolic surfaces. Current Opinion in Solid State and Materials Science 1996, 1, 653-662).

Results

TABLE 9
Liquid crystalline structure results

| Tablet | Structure obtained |
|---|---|
| SBT122 (1:1) | Pn3m |
| SBT127 | Pn3m |
| SBT131 | Pn3m |
| SBT176 (1:1) | Pn3m |
| SBT177 (4:1) | Pn3m |

### Example 8—Formulations with 1:1 w/w Ratio of Statin and Amphiphile (SBT226), Oxycodone and Amphiphile (SBT227) and Adrenaline and Amphiphile (SBT237)

A second statin containing ODT was prepared according to Table 5 above but using 5.42% w/w of atorvastatin calcium trihydrate and the same amount of GMO. The crospovidone was reduced to 10% w/w and the Pharmaburst increased to 68.21% w/w. The ODT (SBT226) had a 1:1 ratio of GMO to atorvastatin calcium trihydrate and 10 mg atorvastatin calcium trihydrate.

A third ODT (SBT227) was prepared according to Table 5 above but using 5% w/w of oxycodone hydrochloride and the same amount of GMO. The crospovidone was also reduced to 10% w/w and the Pharmaburst increased to 69.55% w/w. The ODT (SBT226) had a 1:1 ratio of GMO to oxycodone hydrochloride and 5 mg oxycodone hydrochloride.

A fourth ODT (SBT237) containing 300 μg adrenaline was prepared with a 1:1 ratio of GMO to adrenaline. The formulation is in Table 10 below.

TABLE 10
ODT with 1:1 GMO to adrenaline (SBT237)

| Material Name | % w/w | Notes |
|---|---|---|
| Pharmaburst-(co-processed mixture of | 77.78 | Filler, Taste masking, |
| Mannitol, Sorbitol Crospovidone & | | Disintegration agent. |
| Silicon dioxide) | | |
| Crospovidone XL | 10.68 | Disintegration agent |
| Sodium Chloride | 0.28 | Osmotic agent |
| Sodium Cyclamate | 0.68 | Sweetener |
| Saccharin Sodium | 0.45 | Sweetener |
| Menthol | 0.23 | Flavouring agent |
| Epinephrine (Adrenaline) | 0.34 | Drug substance/API |
| Povidone (Poly vinyl pyrrolidone) | 1.70 | Binder |
| Glyceryl Monooleate (GMO) | 0.34 | Bio adhesive/Mucoadhesive agent, |
| | | Gelling agent, nonionic surfactant, |
| | | sustained release agent |
| Sodium Starch Glycolate | 5.00 | Disintegration agent |
| Colloidal Silicon Dioxide | 1.50 | Glidant |
| Magnesium Stearate | 1.00 | Lubricant |
| Ethanol | N/A * | Solvent |

Each ODT was prepared according to the method in Example 2.

### Example 9—Formulations with 1:4 w/w Ratio of Statin and Amphiphile (SBT233), a 1:4 w/w Ratio Oxycodone and Amphiphile (SBT232) and a 10:1 w/w Ratio of Adrenaline and Amphiphile (SBT238)

A second ODT (SBT233) containing 10 mg atorvastatin was prepared a 4:1 ratio of GMO to atorvastatin. The formulation is in Table 11 below.

TABLE 11
ODT with 4:1 GMO to atorvastatin (SBT233)

| Ingredient | % w/w | Function |
|---|---|---|
| Pharmaburst-(co-processed mixture of | 58.86 | Filler, Taste masking, Disintegration agent. |
| Mannitol, Sorbitol Crospovidone & Silicon | | |
| dioxide) | | |
| Crospovidone XL | 13.82 | Disintegration agent |
| Sodium Chloride | 0.22 | Osmotic agent |
| Sodium Cyclamate | 0.53 | Sweetener |
| Saccharin Sodium | 0.41 | Sweetener |
| Menthol | 0.21 | Flavouring agent |
| Atorvastatin Calcium Trihydrate | 3.19 | Drug substance/API |
| Povidone (Poly vinyl pyrrolidone) | 2.50 | Binder |
| Glyceryl Monooleate (GMO) | 12.76 | Bio adhesive/Mucoadhesive agent, Gelling |
| | | agent, nonionic surfactant, sustained release |
| | | agent |
| Sodium Starch Glycolate | 5.00 | Disintegration agent |
| Colloidal Silicon Dioxide | 1.50 | Glidant |
| Magnesium Stearate | 1.00 | Lubricant |
| Ethanol | N/A * | Solvent |

A third ODT (SBT232) was prepared according to Table 6 but using 1.67% w/w of oxycodone hydrochloride and four times as much GMO (6.67% w/w). The crospovidone was also reduced to 10% w/w and the Pharmaburst increased to 65.22% w/w, when compared to the rosuvastatin formulation in Table 6. The ODT (SBT232) had a 4:1 ratio of GMO to oxycodone hydrochloride and 5 mg oxycodone hydrochloride.

A fourth ODT (SBT238) containing 300 μg adrenaline was prepared with a 10:1 ratio of GMO to adrenaline. The formulation is in Table 12 below.

TABLE 12
ODT with 1:1 GMO to adrenaline (SBT238)

| Material Name | % w/w | Notes |
|---|---|---|
| Pharmaburst-(co-processed mixture of | 70.97 | Filler, Taste masking, Disintegration |
| Mannitol, Sorbitol Crospovidone & | | agent. |
| Silicon dioxide) | | |
| Crospovidone XL | 16.00 | Disintegration agent |
| Sodium Chloride | 0.28 | Osmotic agent |
| Sodium Cyclamate | 0.66 | Sweetener |
| Saccharin Sodium | 0.44 | Sweetener |
| Menthol | 0.22 | Flavouring agent |
| Epinephrine (Adrenaline) | 0.11 | Drug substance/API |
| Povidone (Poly vinyl pyrrolidone) | 2.76 | Binder |
| Glyceryl Monooleate (GMO) | 1.10 | Bio adhesive/Mucoadhesive agent, |
| | | Gelling agent, nonionic surfactant, |
| | | sustained release agent |
| Sodium Starch Glycolate | 4.97 | Disintegration agent |
| Colloidal Silicon Dioxide | 1.49 | Glidant |
| Magnesium Stearate | 0.99 | Lubricant |
| Ethanol | N/A * | Solvent |

Each ODT was prepared according to the method in Example 2.

Disintegration time was tested. The 1:1 oxycodone ODT disintegrated within 20-30 seconds and 4:1 oxycodone ODT disintegrated within 45-70 seconds. The 1:1 adrenaline ODT disintegrated within 15-35 seconds and the 4:1 adrenaline ODT disintegrated within 25-35 seconds.

Liquid crystalline formation was tested using the method of Example 7 but having some ODT samples hydrated for 30 min before testing and some for 18 hours before testing. Each sample was tested at 125 times/locations. The results are in table 13 below. The results were the same for the 30 min and 18 hour hydrated samples.

TABLE 13
Liquid crystalline structure results

| | | Structure | Structure |
|---|---|---|---|
| | | produced-Plate | produced-Plate |
| | | 1: Hydration | 2: Hydration 30 |
| API | Batch number | 18 hrs | minutes |
| Oxycodone 5 mg 1:1 | SBT227 | Cubic Pn3m & | Cubic Pn3m & |
| | | Im3m | Im3m |
| Oxycodone 5 mg 4:1 | SBT232 | Cubic Pn3m | Cubic Pn3m |
| Atorvastatin 10 mg 1:1 | SBT226 | Double Lamellar | Double Lamellar |
| Atorvastatin 10 mg 4:1 | SBT233 | Double Lamellar | Double Lamellar |
| Adrenaline | SBT237 | Hexagonal | Double Lamellar |
| (Epinephrine) 0.3 mg | | | |
| 1:1 | | | |
| Adrenaline | SBT238 | Hexagonal & | Hexagonal |
| (Epinephrine) 0.3 mg | | Cubic Pn3m | |
| 10:1 | | | |
| Rosuvastatin 5 mg 1:1 | SBT176 | Lamellar | Lamellar |
| Rosuvastatin 5 mg 1:1 | SBT187 | Lamellar | Lamellar |
| Rosuvastatin Placebo | SBT189 | Cubic Pn3m | Cubic Pn3m |

*SBT176 was tested when it was over 12 months old. Earlier testing of SBT122, and SBT123, which occurred only a couple months after formulation suggests that SBT176 may have formed form cubic phase if tested closer to its preparation.

A blend of the amphiphilic compound GMO and the active ingredient niacin was shown to form hexagonal phase in international patent publication no. WO 2014/179845.

### Example 10—In Vitro Release Testing

Release of an active ingredient from and ODT through a mucosal membrane can be tested in vitro.

Porcine buccal mucosa was freshly isolated from pigs cheeks, mounted between modified Ussing chambers with a donor chamber, receptor chamber and the porcine buccal mucosa in between with a diffusional area of 0.64 cm², and incubated in Krebs bicarbonate Ringer buffer (KBR, pH 7.4) for 30 min. The tablet was applied to the porcine buccal mucosa (ie in the donor chamber) and, when necessary, Parafilm was applied to cover the formulation (ie for tablets and for mixtures containing glyceryl monooleate (GMO) and rosuvastatin). The Parafilm prevented the various formulations from detaching from the buccal mucosa. KBR buffer (1.5 mL) was then added to both the donor and receptor chambers, and receptor samples (200 μL) were collected from the receptor chamber at various time points up to 4-5 hours to determine the amount of rosuvastatin that passed through the porcine buccal mucosa to the receptor chamber. 200 μL of fresh KBR was dispensed into the receptor chamber after each collection (to ensure volume balance). Receptor chamber samples were quantified by HPLC.

Positive control was tested by making solutions of 0.4 and 0.8 mg/1.5 ml active ingredient in KBR solution, equivalent to 1:1 and 7:1 ODT's.

The permeation of the active ingredient from the ODT of the invention was tested for ODTs containing oxycodone and ODTs containing atorvastatin to establish that the ODT functioned to deliver active ingredients having varied LogP values and varied dosages. This time samples were taken from the receiving chamber of the Ussing chamber repeatedly at 0.5, 1, 1.5, 2, 3 and 4 hours to establish not only that the active ingredient permeated the mucosa but that release of the active ingredient was prolonged.

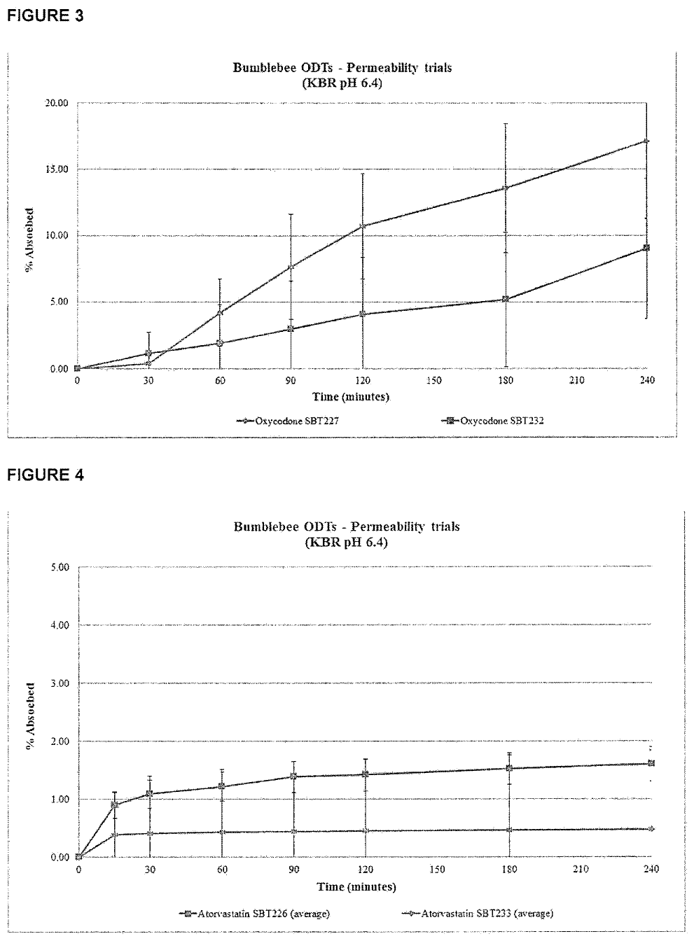

The appearance of oxycodone in the receptor chamber over time is depicted in FIG. 3 after application of ODT SBT227 (diamond) and ODT SBT232 (square) to porcine buccal mucosa in the donor chamber of the Ussing chamber. Data are presented as mean±SEM (n=5). Both formulations demonstrate slow release characteristics. SBT227, with a 1:1 ratio of active ingredient to GMO, released the API relatively faster compared to SBT232, with a 1:4 ratio of active ingredient to GMO. Without being bound by theory, this is thought to be due to the lower quantity of GMO in the SBT227 formulation.

The appearance of atorvastatin in the receptor chamber over time is depicted in FIG. 4 after application of ODT SBT226 (square) and SBT233 (diamond) to porcine buccal mucosa in the donor chamber of the Ussing chamber. Data are presented as mean±SEM (n=5). Both formulations demonstrate slow release characteristics. SBT226, with a 1:1 ratio of active ingredient to GMO, released the API relatively faster comparing to SBT233, with a 1:4 ratio of active ingredient to GMO. Without being bound by theory, this is thought to be due to the lower quantity of GMO in the SBT226 formulation.

### Example 11—Prolonged Release

FIG. 3 shows that in vitro release of oxycodone through a porcine mucosal membrane shows a prolonged release profile with release continuing steadily at 4 hours. Maximum blood concentration is achieved about 1 hour after administration of an OxyNorm tablet.

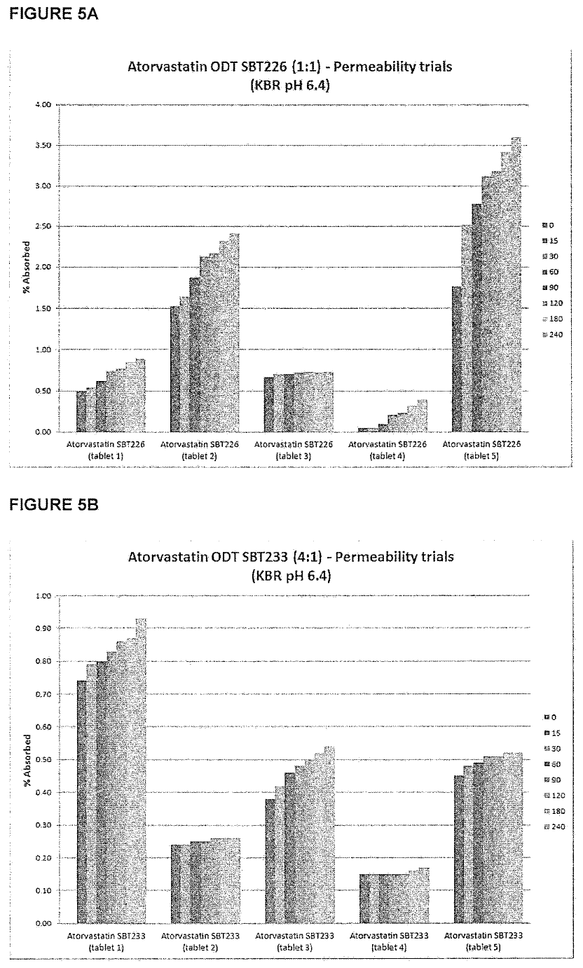

Similarly, FIGS. 5A and 5B shows that in vitro release of atorvastatin through a porcine mucosal membrane was slow release and continuing at 4 hours for 4 of the 5 SBT226 and 3 of the 5 SBT233 tablets tested. Without being bound by theory, Ussing chamber testing is less robust than some in vitro testing methods and it is possible that there was a technical difficulty in the testing of the tablets that did not show slow release. Lipitor oral tablets achieve maximum plasma concentration within 1-2 hours following administration.

A blend of the amphiphilic compound GMO and the active ingredient niacin was shown to exhibit prolonged release in international patent publication no. WO 2014/179845.

1. An oral disintegrating tablet (ODT) suitable for systemic administration of an active ingredient via the oral mucosa comprising an active ingredient, about 1 to about 20% w/w glycerol monooleate, about 1 to about 60% w/w of at least one disintegrant and about 0.5 to about 5% w/w of at least one binder, and wherein, when the ODT contacts a hydrophilic solvent, the ODT disintegrates and the amphiphilic compound self-assembles into liquid crystalline particles.

2. The ODT according to claim 1, wherein the ODT has hardness of at least about 0.5 to about 6 kp.

3. The ODT according to claim 1, wherein the hardness is about 1 to about 4 kp.

4-5. (canceled)

6. The ODT according to claim 1, wherein the liquid crystalline particles are cubosomes.

7-8. (canceled)

9. The ODT according to claim 1, wherein the ODT disintegrates within 15 minutes of contact with a hydrophilic solvent.

10. The ODT according to claim 1, wherein the ODT is formulated for sublingual or buccal administration.

11. The ODT according to claim 1, wherein the active ingredient has a log P of −0.5 to 6.4 and/or a molecular weight of 100 to 1200.

12. The ODT according to claim 1, wherein the active ingredient is 0.5 to 10% w/w of the ODT.

13. The ODT according to claim 1, wherein the amphiphilic compound has one or more of the group selected from a critical packing parameter (CPP) of >½ and a hydrophilic lipophilic balance (HLB) of 0 to <10.

14. (canceled)

15. The ODT according to claim 1, wherein when the ODT disintegrates upon contact with a hydrophilic solvent the amphiphilic compound self-assembles into liquid crystalline particles that encapsulate or entrain the active ingredient.

16. A method of preparing an ODT comprising
heating an amphiphilic compound capable of self-assembly into liquid crystalline particles upon contact with a hydrophilic solvent to its melting point
mixing an active ingredient with the amphiphilic compound until dispersed
cooling the mixture to at least a semi-solid state
combining the mix of active ingredient and amphiphilic compound with at least one pharmaceutically acceptable excipient to prepare a blend
optionally adding further pharmaceutically acceptable excipients
compressing the blend into an ODT
wherein, when the ODT contacts a hydrophilic solvent the ODT disintegrates and the amphiphilic compound self-assembles into liquid crystalline particles.

17. The method of claim 16, wherein combining the mix of active ingredient and amphiphilic compound with at least one further excipient reduces the temperature of the amphiphilic compound to below its melting point.

18. The method of claim 16, wherein the at least one pharmaceutically acceptable excipient is added immediately following mixing of the amphiphilic compound and active ingredient.

19. The method of claim 16, wherein mixing of the amphiphilic compound and active ingredient occurs within 10 minutes.

20. The method of claim 16, wherein the amphiphilic compound is not heated more than 10° C. above its melting point.

21. A method of preparing an ODT comprising
heating an amphiphilic compound capable of self-assembly into liquid crystalline particles upon contact with a hydrophilic solvent to its melting point
mixing an active ingredient with the amphiphilic compound until dispersed immediately following melting of the amphiphilic compound or alternatively during melting the amphiphilic compound
cooling the mixture to at least a semi-solid state immediately following dispersion of the active ingredient within the amphiphilic compound
combining the mix of active ingredient and amphiphilic compound with at least one pharmaceutically acceptable excipient
optionally adding further pharmaceutically acceptable excipients
compressing the blend into an ODT
wherein, when the ODT contacts a hydrophilic solvent the ODT disintegrates and the amphiphilic compound self-assembles into liquid crystalline particles.

22. The method of claim 16, wherein the ODT has a hardness of about 0.5 to about 6 kp.

23. The method of claim 16, wherein the ODT disintegrates within 15 minutes of contact with a hydrophilic solvent.

24. The method of claim 16, wherein the liquid crystalline particles are cubosomes.

25. The method of claim 16, wherein the ODT is formulated for sublingual or buccal administration.
